(19) United States
(12) Patent Application Publication
(10) Pub. No.: US 2013/0123282 A1
(43) Pub. Date: May 16, 2013

(54) SOLID STATE FORMS OF LINAGLIPTIN

(71) Applicants: Leonid Metsger, Beer-Sheva (IL); Revital Burstein, Ramat-Hasharon (IL); Ariel Mitteman, Elad (IL); Motti Erlich, Petach-Tikva (IL); Slavik Yurkovsky, Kiryat-Gat (IL)

(72) Inventors: Leonid Metsger, Beer-Sheva (IL); Revital Burstein, Ramat-Hasharon (IL); Ariel Mitteman, Elad (IL); Motti Erlich, Petach-Tikva (IL); Slavik Yurkovsky, Kiryat-Gat (IL)

(21) Appl. No.: 13/678,354

(22) Filed: Nov. 15, 2012

Related U.S. Application Data
(60) Provisional application No. 61/560,374, filed on Nov. 16, 2011.
(60) Provisional application No. 61/565,077, filed on Nov. 30, 2011.
(60) Provisional application No. 61/569,397, filed on Dec. 12, 2011.
(60) Provisional application No. 61/576,184, filed on Dec. 15, 2011.
(60) Provisional application No. 61/581,231, filed on Dec. 29, 2011.
(60) Provisional application No. 61/592,086, filed on Jan. 30, 2012.
(60) Provisional application No. 61/599,688, filed on Feb. 16, 2012.
(60) Provisional application No. 61/605,347, filed on Mar. 1, 2012.
(60) Provisional application No. 61/637,571, filed on Apr. 24, 2012.
(60) Provisional application No. 61/647,805, filed on May 16, 2012.
(60) Provisional application No. 61/712,011, filed on Oct. 10, 2012.

(51) Int. Cl. C07D 473/04

(52) U.S. Cl. CPC C07D 473/04
USPC 514/263.21; 544/270

(57) ABSTRACT

The present invention provides solid state forms of Linagliptin, processes for preparing the solid state forms, and pharmaceutical compositions thereof.

### CROSS REFERENCE TO RELATED APPLICATIONS

This application claims the benefit of U.S. Provisional Application Nos. 61/560,374, filed Nov. 16, 2011, 61/565,077, filed Nov. 30, 2011, 61/569,397, filed Dec. 12, 2011, 61/576,184, filed Dec. 15, 2011, 61/581,231, filed Dec. 29, 2011, 61/592,086, filed Jan. 30, 2012, 61/599,688, filed Feb. 16, 2012, 61/605,347, filed Mar. 1, 2012, 61/637,571, filed Apr. 24, 2012, 61/647,805, filed May 16, 2012, and 61/712,011, filed Oct. 10, 2012, the contents of which are incorporated herein by reference.

### FIELD OF THE INVENTION

The invention relates to solid state forms of Linagliptin, processes for preparing the solid state forms, and pharmaceutical compositions thereof.

### BACKGROUND OF THE INVENTION

Linagliptin, 8-[3(R)-aminopiperidin-1l-yl]-7-(2-butynyl)-3-methyl-1-(4-methyl quinazolin-2-ylmethyl)xanthine, has the following chemical structure:

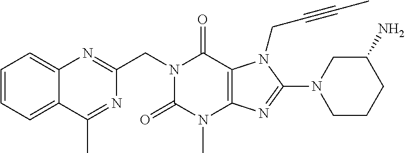 [SMILES: CC#CCn1c(N2CCC[C@@H](N)C2)nc2c1c(=O)n(Cc1nc(C)c3ccccc3n1)c(=O)n2C]

It is a DPP-4 inhibitor developed by Boehringer Ingelheim for the treatment of type II diabetes.

PCT Publications WO 2004/018468 and WO 2006/048427 describe synthesis of Linagliptin. Crystalline forms of Linagliptin, Forms A, B, C, D, and E are described in the PCT Publication No. WO 2007/128721. According to WO 2007/128721, Linagliptin prepared according to Publication No. WO 2004/018468 is present in ambient temperature as a mixture of two enantiotropic polymorphs. The temperature at which the two polymorphs transform into one another is 25±15° C. The pure high temperature form (polymorph A), can be obtained by heating the mixture to temperatures>40° C. The low temperature form (polymorph B) is obtained by cooling to temperatures<10° C.”. According to WO 2007/128721, the transition point between forms A and B is at room temperature, such that they exist as a polymorphic mixture. In addition, WO 2007/128721 teaches that form D “is obtained if polymorph C is heated to a temperature of 30-100° C. or dried at this temperature”. Since the procedure to obtain form C according to this application includes drying at 70° C., the dried form C is expected to be obtained in admixture with form D. WO 2007/128721 teaches that Form E is obtained only at high temperatures (after melting of form D at 150±3° C.), and therefore is not relevant industrially. There is a need for new polymorphs of Linagliptin that have better characteristics, for example, can be obtained and exist at room temperature as single, pure phase.

Polymorphism, the occurrence of different crystal forms, is a property of some molecules and molecular complexes. A single molecule may give rise to a variety of polymorphs having distinct crystal structures and physical properties like melting point, thermal behaviors (e.g., measured by thermogravimetric analysis—“TGA”, or differential scanning calorimetry—“DSC”), powder X-ray diffraction (XRD) pattern, infrared absorption fingerprint, and solid state NMR spectrum. One or more of these techniques may be used to distinguish different polymorphic forms of a compound.

Discovering new polymorphic forms and solvates of a pharmaceutical product can provide materials having desirable processing properties, such as ease of handling, ease of processing, storage stability, and ease of purification or as desirable intermediate crystal forms that facilitate conversion to other polymorphic forms. New polymorphic forms and solvates of a pharmaceutically useful compound or salts thereof can also provide opportunities to improve the performance characteristics of a pharmaceutical product. They can also enlarge the repertoire of materials available to a formulation scientist for formulation optimization, for example by providing a product with different properties, e.g., better processing or handling characteristics, improved dissolution profile, or improved shelf-life. For at least these reasons, there is a need for additional polymorphs of Linagliptin.

### SUMMARY OF THE INVENTION

The present invention provides new solid state forms of Linagliptin. These solid state forms can be used to prepare Linagliptin salts and/or formulations thereof.

The invention further provides the solid state forms of Linagliptin as described below for use in the manufacture of a medicament for the treatment of type II diabetes; and provides a method of treating type II diabetes, said method comprising administering a therapeutically effective dose of one or more of the solid state forms described herein.

### BRIEF DESCRIPTION OF THE FIGURES

FIG. 1 provides a powder XRD pattern of crystalline Form I of Linagliptin.

FIG. 2 provides a powder XRD pattern of crystalline Form II of Linagliptin.

FIG. 3 provides a powder XRD pattern of Form III of Linagliptin.

FIG. 4 provides a powder XRD pattern of crystalline Form IV of Linagliptin.

FIG. 5 provides a powder XRD pattern of Form V of Linagliptin.

FIG. 6 provides a powder XRD pattern of crystalline Form VI of Linagliptin.

FIG. 7 provides a powder XRD pattern of crystalline Form VII of Linagliptin.

FIG. 8 provides a powder XRD pattern of crystalline Form VIII of Linagliptin.

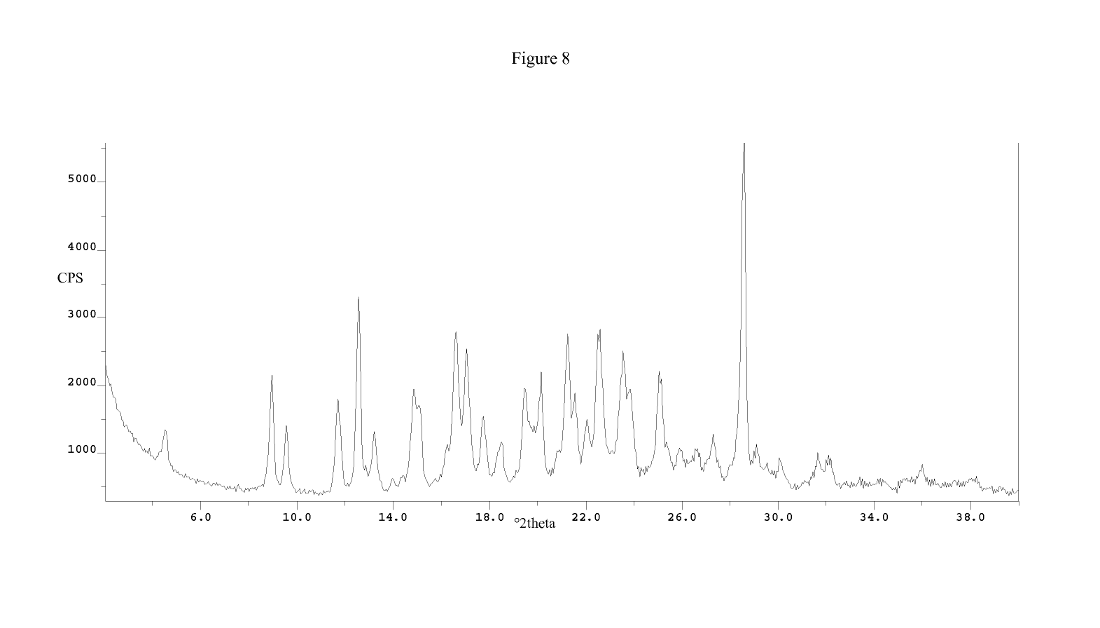

FIG. 9 provides a powder XRD pattern of crystalline Form IX of Linagliptin.

FIG. 10 provides a powder XRD pattern of crystalline Form X of Linagliptin.

FIG. 11 provides a powder XRD pattern of crystalline Form XI of Linagliptin.

FIG. 12 provides a powder XRD pattern of crystalline Form XI′ of Linagliptin.

FIG. 13 provides a powder XRD pattern of crystalline Form XII of Linagliptin.

FIG. 14 provides a powder XRD pattern of crystalline Form XIII of Linagliptin.

FIG. 15 provides a powder XRD pattern of crystalline Form XIV of Linagliptin.

FIG. 16 provides a powder XRD pattern of crystalline Form XV of Linagliptin.

FIG. 17 provides a powder XRD pattern of crystalline Form XIII′ of Linagliptin.

FIG. 18 provides a powder XRD pattern of crystalline Form XVI of Linagliptin.

FIG. 19 provides a powder XRD pattern of crystalline Form VIII′ of Linagliptin.

FIG. 20 provides a powder XRD pattern of crystalline Form XVIII of Linagliptin.

FIG. 21 provides a powder XRD pattern of crystalline Form XVII of Linagliptin.

FIG. 22 provides a powder XRD pattern of crystalline Form XIX of Linagliptin.

FIG. 23 provides a powder XRD pattern of crystalline Form XX of Linagliptin.

FIG. 24 provides a powder XRD pattern of crystalline Form XXI of Linagliptin.

FIG. 25 provides a powder XRD pattern of crystalline Form XXII of Linagliptin.

FIG. 26 provides a powder XRD pattern of crystalline Form XXIII of Linagliptin.

FIG. 27 provides a powder XRD pattern of crystalline Form XXII, as obtained in example 32.

FIG. 28 provides a powder XRD pattern of crystalline Form XXIV.

FIG. 29 provides a ¹³C NMR spectrum of Form X in the range of 0-180 ppm.

FIG. 30 provides a ¹³C NMR spectrum of Form X in the range of 100-180 ppm.

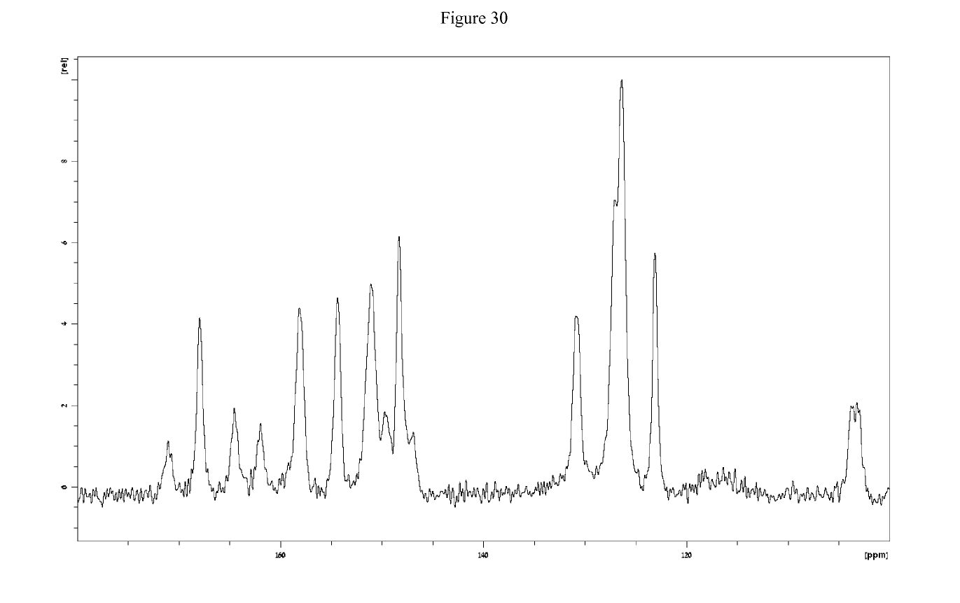

### DETAILED DESCRIPTION OF THE INVENTION

The present invention provides new solid state forms of Linagliptin. These solid state forms can be used to prepare salts and/or formulations thereof.

The solid state forms of the present invention exist as single phase at room temperature. In the pharmaceutical industry there is a need for single-phase stable forms that do not transform into other forms at ambient conditions, and may be formulated even after prolonged storage times, without the risk of having a polymorphic conversion before and after formulation.

In addition, the solid state forms of the present invention have advantageous properties selected from at least one of: chemical purity, flowability, solubility, morphology or crystal habit, stability—such as storage stability, stability to dehydration, stability to polymorphic conversion, low hygroscopicity, and low content of residual solvents.

A polymorph may be referred to herein as substantially free of any other solid forms. As used herein in this context, the expression “substantially free” will be understood to mean that the solid state form contains 20% or less, 10% or less, 5% or less, 2% or less, or 1% or less of any other solid form of the subject compound as measured, for example, by powder X-ray diffraction (PXRD). Thus, polymorphs of Linagliptin described herein as substantially free of any other solid forms would be understood to contain greater than 80% (w/w), greater than 90% (w/w), greater than 95% (w/w), greater than 98% (w/w), or greater than 99% (w/w) of the subject form of Linagliptin. Accordingly, in some embodiments of the invention, the described polymorphs of Linagliptin may contain from 1% to 20% (w/w), from 5% to 20% (w/w), or from 5% to 10% (w/w) of one or more other solid forms of Linagliptin.

A solid state form may be referred to herein as being characterized by graphical data “as shown in” a Figure. Such data include, for example, powder X-ray diffractograms and solid state NMR spectra. The skilled person will understand that such graphical representations of data may be subject to small variations, e.g., in peak relative intensities and peak positions due to factors such as variations in instrument response and variations in sample concentration and purity, which factors are well known to the skilled person. Nonetheless, the skilled person would readily be capable of comparing the graphical data in the Figures herein with graphical data generated for an unknown crystal form and confirming whether the two sets of graphical data characterize the same solid state form or two different solid state forms. The skilled person would understand that a solid state form referred to herein as being characterized by graphical data “as shown in” a Figure would include any solid state form of the same chemical characterized by graphical data substantially similar to the Figure except for such small variations, the potential occurrence of which is well known to the skilled person.

Unless indicated otherwise, the solid state forms of the present invention can be dried. Drying may be carried out, for example, at elevated temperature under reduced pressure. The crystalline form can be dried, for example, at a temperature from about 40° C. to about 80° C., or about 40° C. to about 50° C., for example, about 40° C. The drying can be carried out under reduced pressure (i.e., less then 1 atmosphere, for example, about 10 mbar to about 100 mbar, or about 10 mbar to about 25 mbar). The drying can be carried out over a suitable period, for example, of about 8 hours to about 36 hours, or about 10 hours to about 24 hours, for example, about 16 hours. Drying can be carried out overnight.

As used herein, and unless stated otherwise, the term “anhydrous” in relation to crystalline Linagliptin relates to a crystalline Linagliptin which contains not more than 1% (w/w), more preferably not more than 0.5% (w/w) of either water or organic solvents as measured by KF or TGA.

As used herein, and unless stated otherwise, the term “non-hygroscopic” in relation to crystalline Linagliptin refers to less than 0.2% (w/w) absorption of water, for example atmospheric water, by the crystalline Linagliptin, as determined according to Ph.Eur. chapter 5.11 (“Hygroscopicity”)

As used herein, the term “Form A” refers to Linagliptin Form A, which is mentioned in WO 2007/128721. This polymorph can be characterized by XRD peaks at 11.49 Å, 7.60 Å, 7.15 Å, 3.86 Å, 3.54 Å, and 3.47 Å. This form can be prepared, for example, according to the process described in WO 2007/128721.

As used herein, the term “Form B” refers to Linagliptin Form B, which is mentioned in WO 2007/128721. This polymorph can be characterized by XRD peaks at 11.25 Å, 9.32 Å, 7.46 Å, 6.98 Å, and 3.77 Å. This form can be prepared, for example, according to the process described in WO 2007/128721.

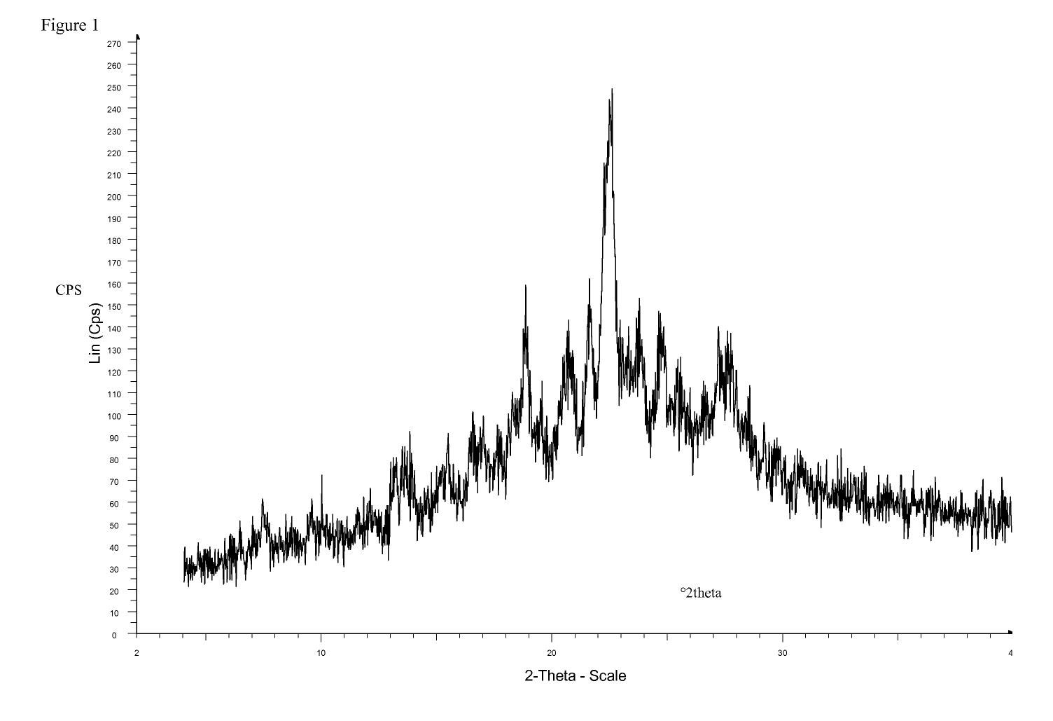

The present invention provides a crystalline Linagliptin, designated Form I. Form I can be characterized by data selected from: a powder XRD pattern with peaks at 18.9, 21.7, 22.6, 23.8 and 24.8±0.3 degrees 2θ; a powder XRD pattern as shown in FIG. 1; and any combinations thereof.

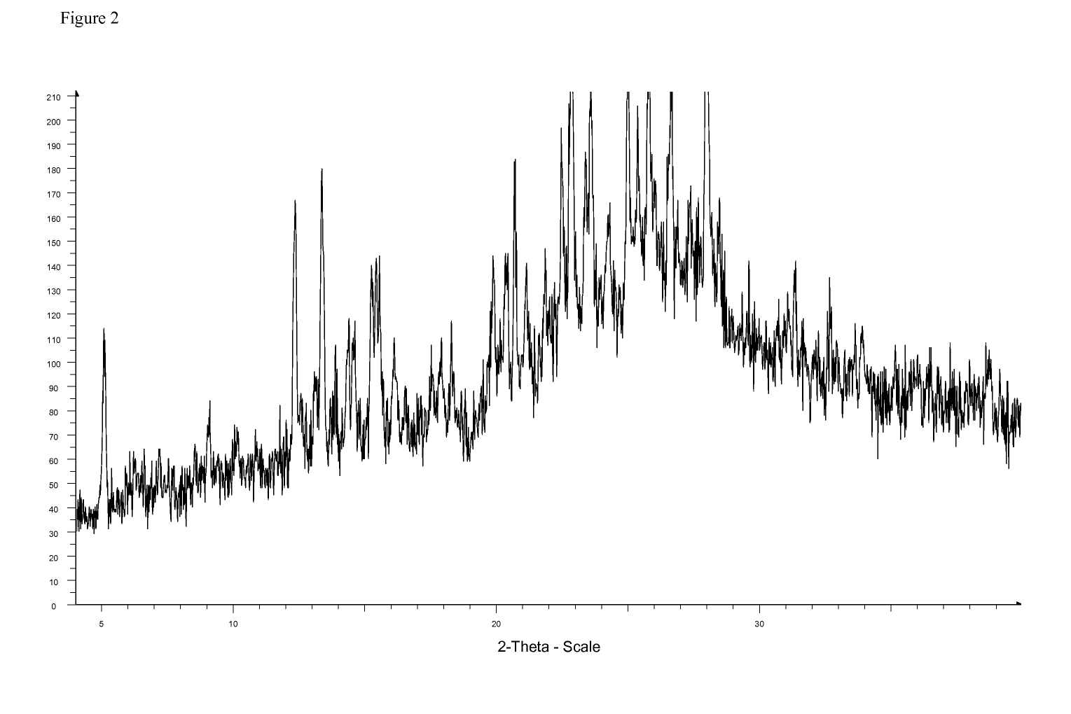

The present invention also provides a crystalline Linagliptin, designated Form II. Form II can be characterized by data selected from: a powder XRD pattern having peaks at 5.1°, 12.3°, 13.4°, 20.7°, and 22.8°±0.2 degrees 2θ; a powder XRD pattern as shown in FIG. 2; and any combinations thereof.

Alternatively, Form II can be characterized by a powder XRD pattern having peaks at 5.1°, 12.3°, 13.4°, 20.7°, and 22.8°±0.2 degrees 2θ, and also having any one, two, three, four or five peaks selected from 23.6°, 24.9°, 25.8°, 26.6°, and 28.0°±0.2 degrees 2θ.

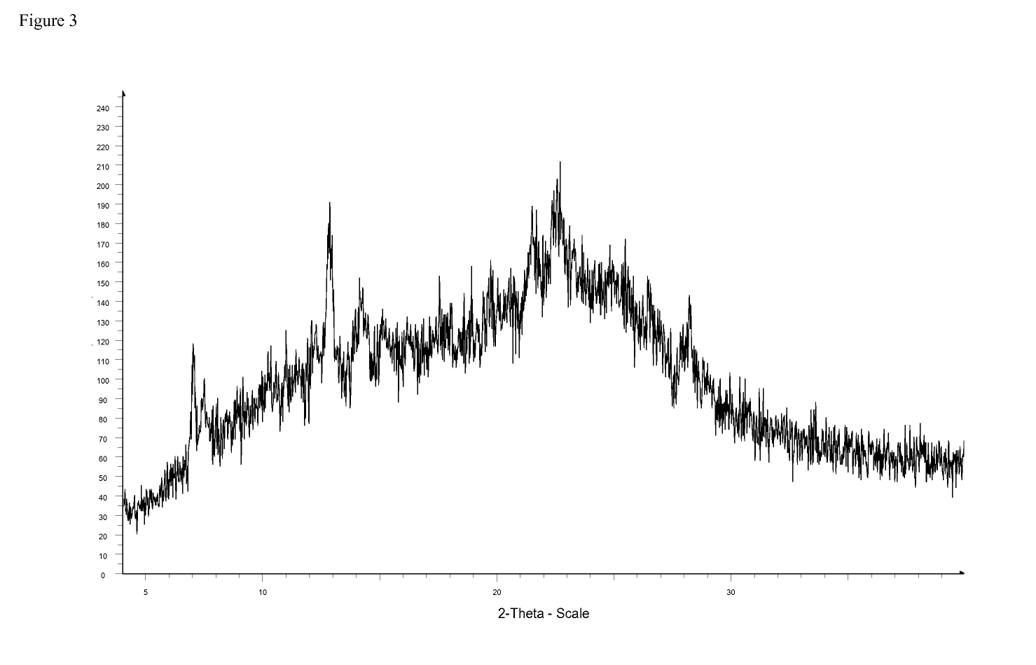

The present invention also provides a form of Linagliptin, designated Form III. Form III can be characterized by data selected from: a powder XRD pattern having peaks at 7.0°, 12.8°, 14.1°, 21.5°, and 22.6°±0.2 degrees 2θ; a powder XRD pattern as shown in FIG. 3; and any combinations thereof.

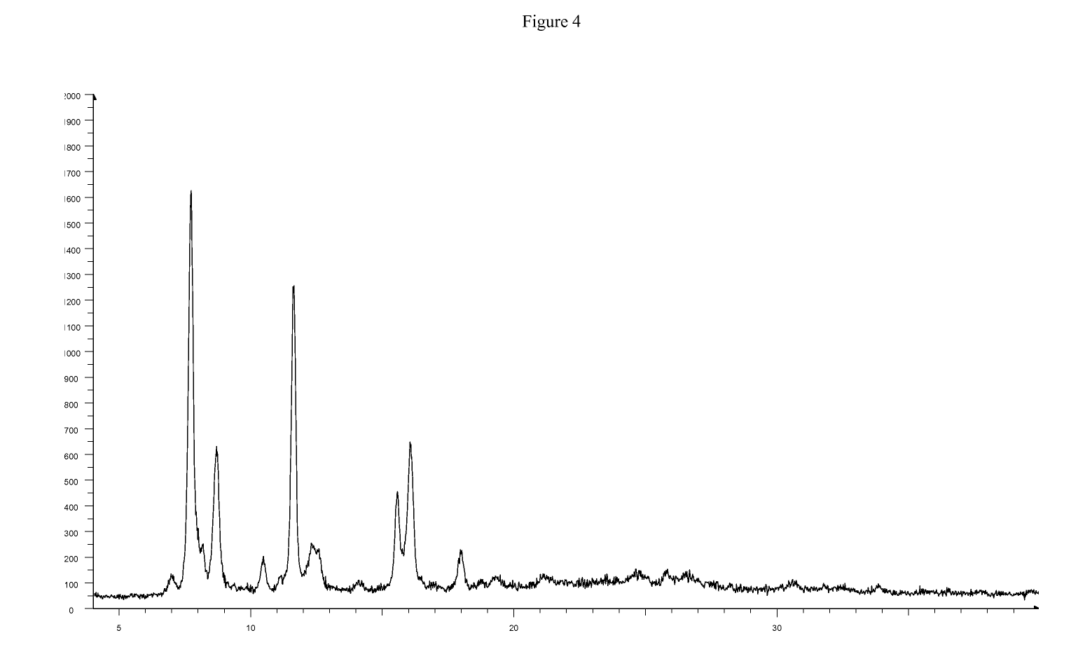

The present invention also provides a crystalline Linagliptin, designated Form IV. Form IV can be characterized by data selected from: a powder XRD pattern having peaks at 6.9°, 7.7°, 8.2°, 8.7°, and 10.5°±0.2 degrees 2θ; a powder XRD pattern as shown in FIG. 4; and any combinations thereof.

Alternatively, Form IV can be characterized by a powder XRD pattern having peaks at 6.9°, 7.7°, 8.2°, 8.7°, and 10.5°±0.2 degrees 2θ, and also having any one, two, three, or four peaks selected from 11.6°, 15.5°, 16.0°, and 18.0°±0.2 degrees 2θ.

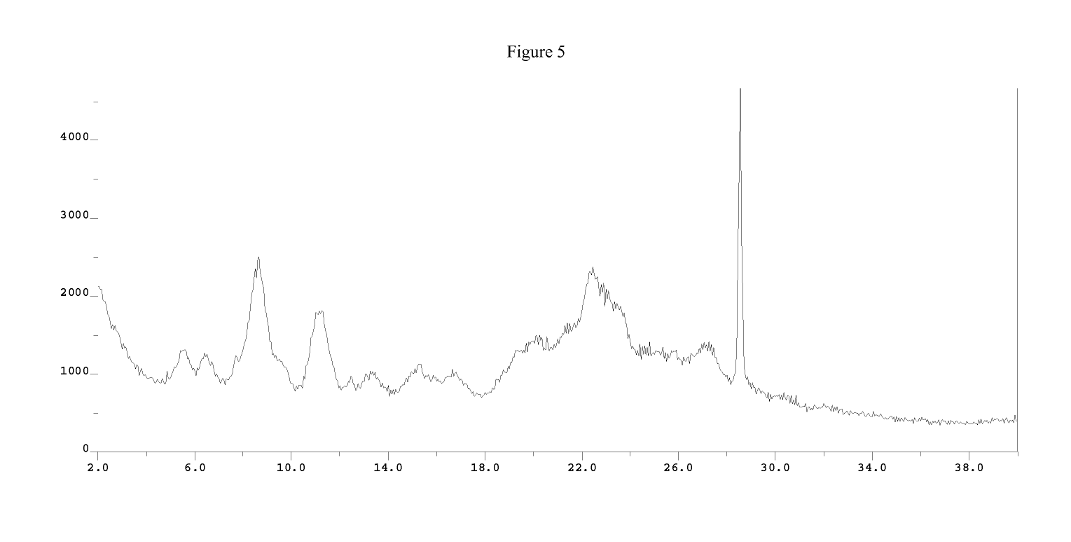

The present invention also provides a form of Linagliptin, designated Form V. Form V can be characterized by data selected from: a powder XRD pattern having peaks at 5.5°, 6.4°, 8.7°, 11.2°, 22.4°±0.2 degrees 2θ; a powder XRD pattern as shown in FIG. 5; and any combinations thereof.

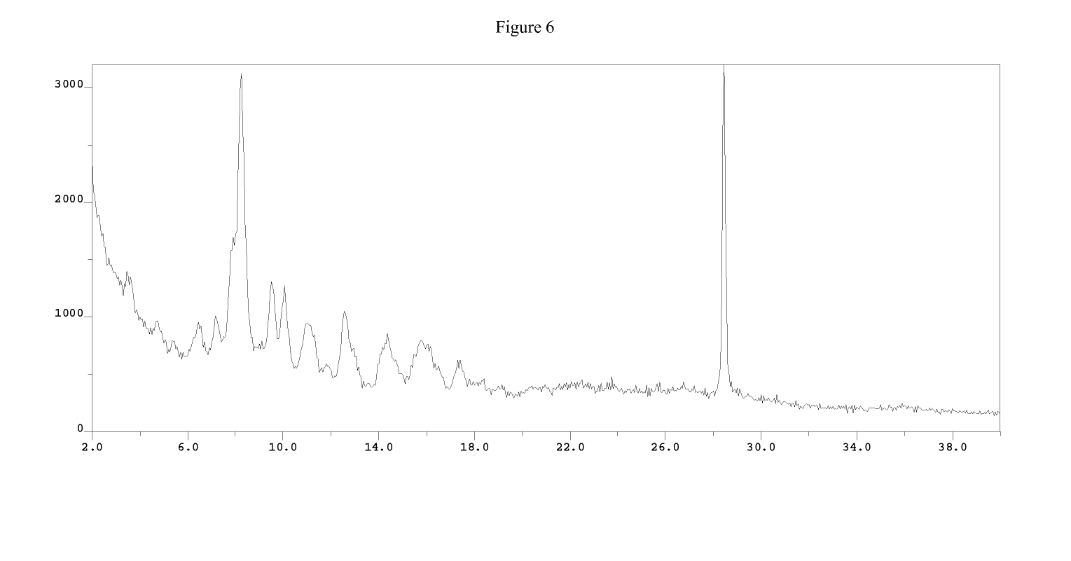

The present invention also provides a crystalline Linagliptin, designated Form VI. Form VI can be characterized by data selected from: a powder XRD pattern having peaks at 6.5°, 7.1°, 8.2°, 9.5°, and 10.1°±0.2 degrees 2θ; a powder XRD pattern as shown in FIG. 6; and any combinations thereof.

Alternatively, Form VI can be characterized by a powder XRD pattern having peaks at 6.5°, 7.1°, 8.2°, 9.5°, and 10.1°±0.2 degrees 2θ, and also having any one, two, three, or four peaks selected from 12.6°, 14.4°, 15.8°, and 17.4°±0.2 degrees 2θ.

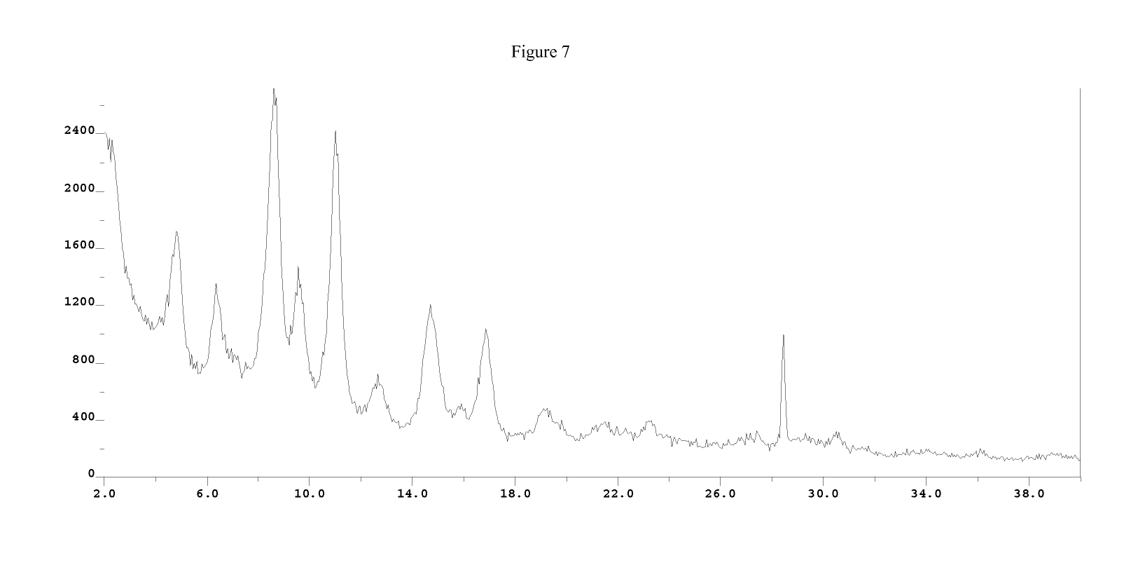

The present invention also provides a crystalline Linagliptin, designated Form VII. Form VII can be characterized by data selected from: a powder XRD pattern having peaks at 4.8°, 6.4°, 8.6°, 9.5°, and 11.0°±0.2 degrees 2θ; a powder XRD pattern as shown in FIG. 7; and any combinations thereof.

Alternatively, Form VII can be characterized by a powder XRD pattern having peaks at 4.8°, 6.4°, 8.6°, 9.5°, and 11.0°±0.2 degrees 2θ, and also having any one, two, three, or four peaks selected from 12.6°, 14.7°, 16.9°, and 19.2°±0.2 degrees 2θ.

The present invention also provides a crystalline Linagliptin, designated Form VIII. Form VIII can be characterized by data selected from: a powder XRD pattern having peaks at 8.9°, 11.7°, 12.5°, 16.6°, and 17.0°±0.2 degrees 2θ; a powder XRD pattern as shown in FIG. 7; and any combinations thereof.

Alternatively, Form VIII can be characterized by a powder XRD pattern having peaks at 8.9°, 11.7°, 12.5°, 16.6°, and 17.0°±0.2 degrees 2θ, and also having any one, two, three, four, or five peaks selected from 19.5°, 20.1°, 21.2°, 22.5°, and 25.1°±0.2 degrees 2θ.

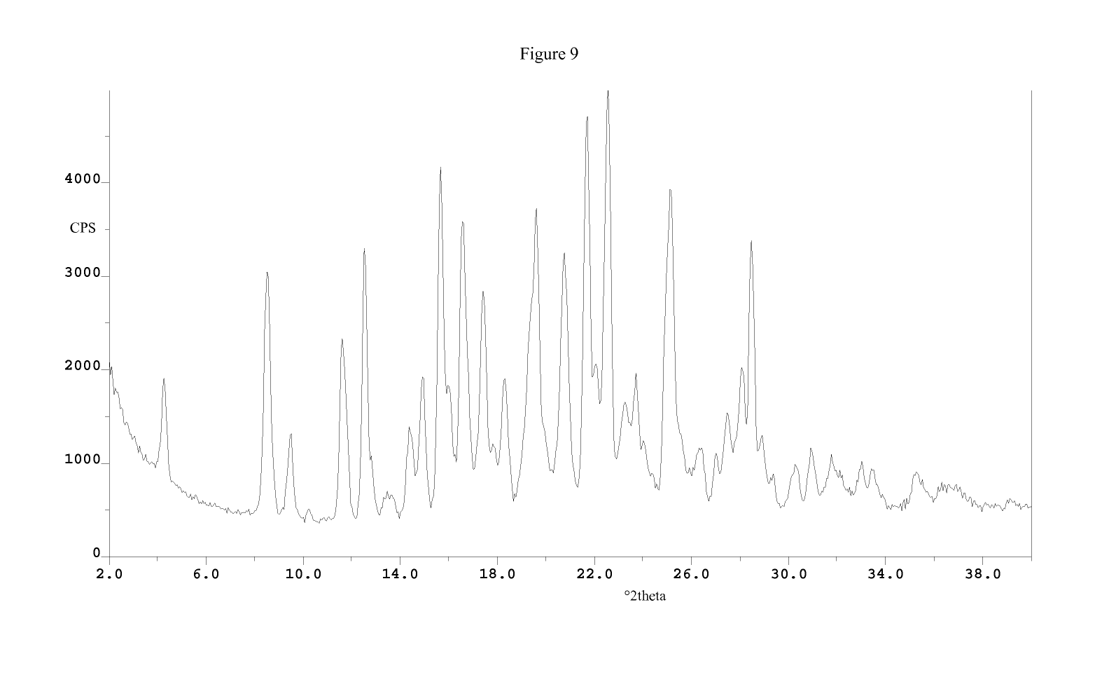

The present invention also provides a crystalline Linagliptin, designated Form IX. Form IX can be characterized by data selected from: a powder XRD pattern having peaks at 4.3°, 15.6°, 19.5°, 20.7°, and 21.7°±0.2 degrees 2θ; a powder XRD pattern as shown in FIG. 9; and any combinations thereof.

Alternatively, Form IX can be characterized by a powder XRD pattern having peaks at 4.3°, 15.6°, 19.5°, 20.7°, and 21.7°±0.2 degrees 2θ, and also having any one, two, three, four, or five peaks selected from 8.5°, 16.6°, 17.4°, 22.5°, and 25.1°±0.2 degrees 2θ.

The present invention also provides a crystalline Linagliptin, designated Form X. Form X can be anhydrous, i.e. it can have a water/organic solvent content of between about 0.01% to about 1% w/w as measured by GC or by KF.

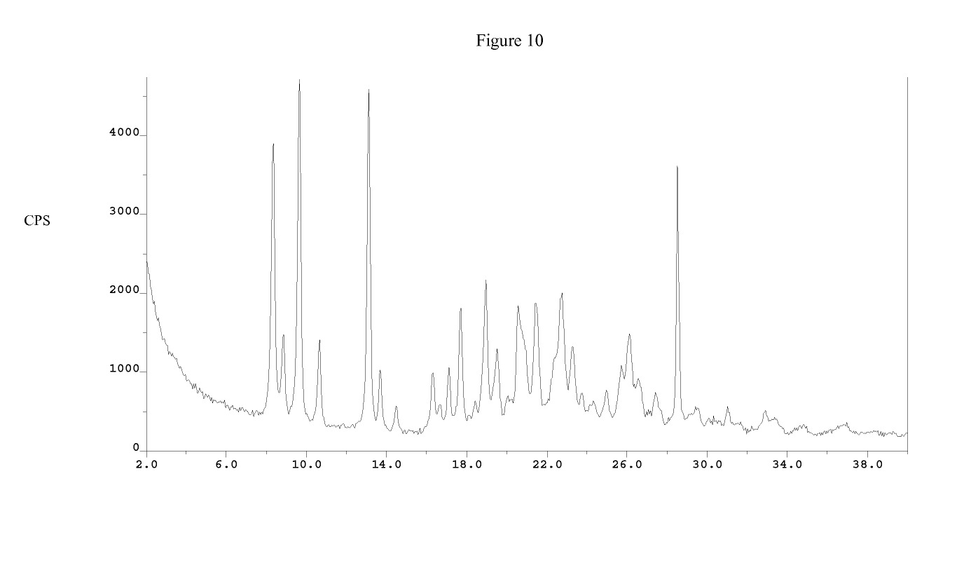

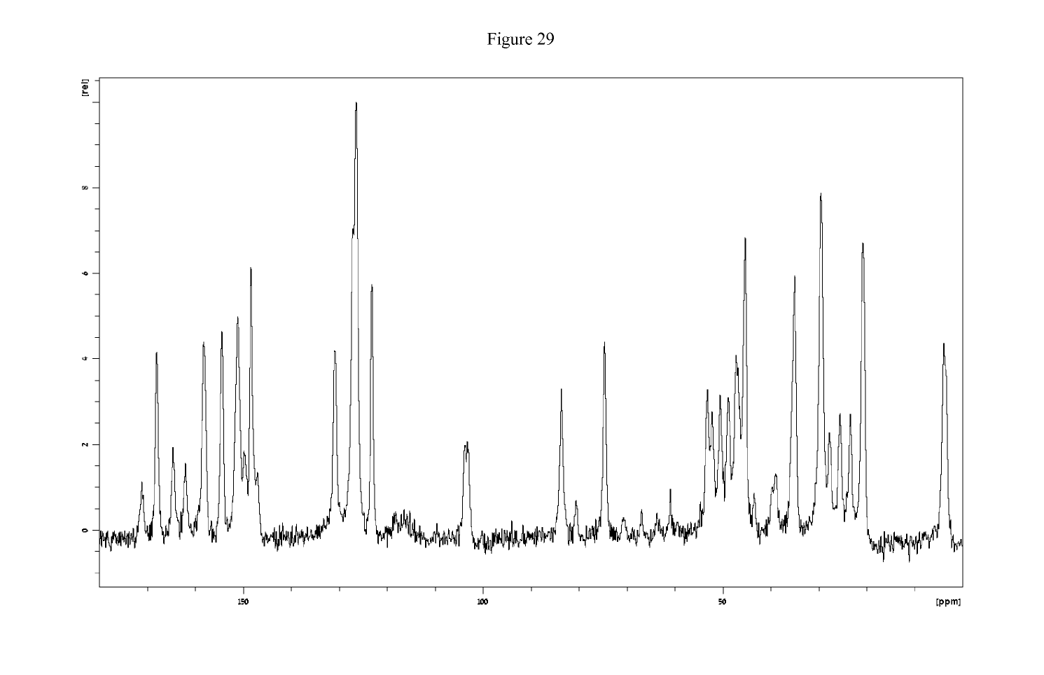

Form X can be characterized by data selected from: a powder XRD pattern having peaks at 8.3°, 9.6°, 13.0°, 17.6°, and 18.9°±0.2 degrees 2θ; a powder XRD pattern as shown in FIG. 10; a solid state ¹³C NMR spectrum having peaks at 126.4, 130.9 and 158.1±0.2 ppm; a solid state ¹³C NMR spectrum having chemical shift differences between the signal exhibiting the lowest chemical shift and another in the chemical shift range of 100 to 180 ppm of about 19.9, 23.2 and 45.1±0.2 ppm; a ¹³C NMR spectrum as shown in FIG. 29; and any combinations thereof; wherein, the signal exhibiting the lowest chemical shift in the chemical shift area of 90 to 180 ppm is at about 103.2±1 ppm.

Alternatively, Form X can be characterized by a powder XRD pattern having peaks at 8.3°, 9.6°, 13.0°, 17.6°, and 18.9°±0.2 degrees 2θ, and also having any one, two, three, four, or five peaks selected from 8.8°, 10.6°, 13.6°, 16.2°, and 17.0°±0.2 degrees 2θ.

In a specific embodiment, the invention encompasses Form X, as described above, can be non-hygroscopic.

Form X can be prepared, for example, by a process comprising slurrying amorphous Linagliptin in ethyl acetate.

In addition to the properties mentioned above, Form XXII is particularly important as it can be obtained and exist at ambient conditions as single, pure phase. Form X is also particularly advantageous, for example, in being non-hygroscopic and polymorphic stable under different relative humidity levels. For example, when stored for 7 days at room temperature under 0-80% relative humidity, no polymorphic transformation is observed.

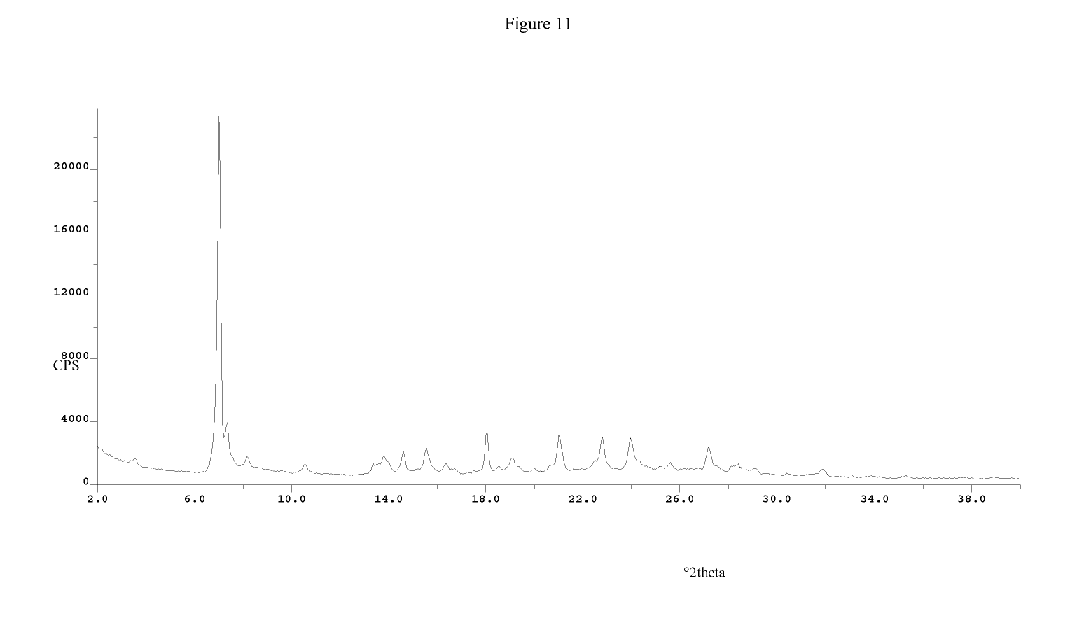

The present invention also provides a crystalline Linagliptin, designated Form XI. Form XI can be characterized by data selected from: a powder XRD pattern having peaks at 17.0°, 18.0°, 21.0°, 22.8°, and 23.9°±0.2 degrees 2θ; a powder XRD pattern as shown in FIG. 11; and any combinations thereof.

Alternatively, Form XI can be characterized by a powder XRD pattern having peaks at 17.0°, 18.0°, 21.0°, 22.8°, and 23.9°±0.2 degrees 2θ, and also having any one, two, three, four, or five peaks selected from 7.3°, 8.2°, 14.6°, 15.5°, and 27.2°±0.2 degrees 2θ.

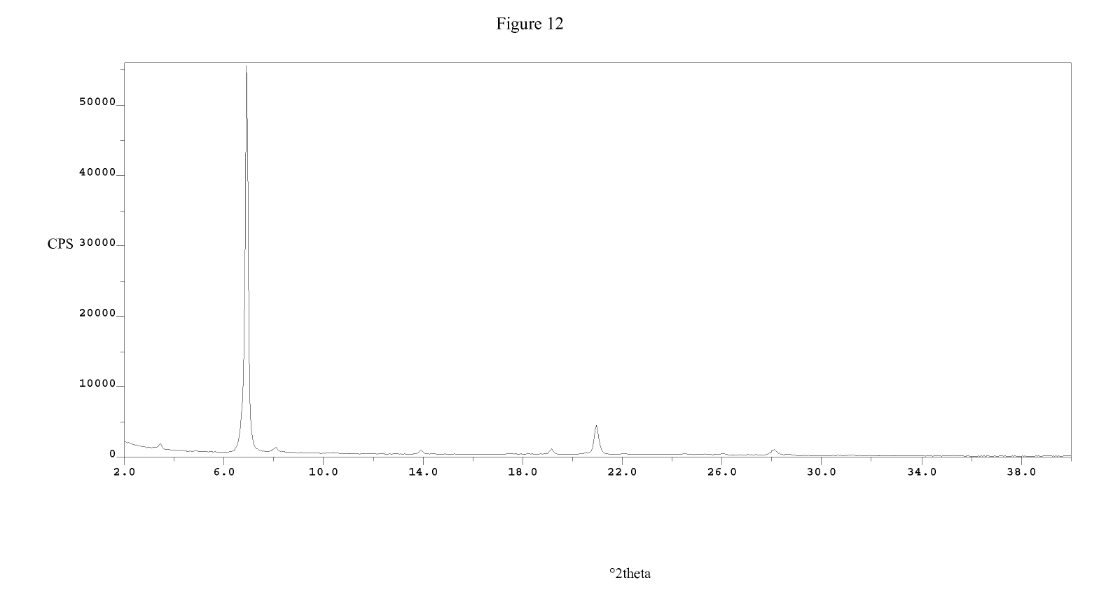

The present invention also provides a crystalline Linagliptin, designated Form XI′. Form XI′ can be characterized by data selected from: a powder XRD pattern having peaks at 6.9°, 8.1°, 13.9°, 19.1°, and 20.9°±0.2 degrees 2θ; a powder XRD pattern as shown in FIG. 12; and any combinations thereof.

Alternatively, Form XI′ can be characterized by a powder XRD pattern having peaks at 6.9°, 8.1°, 13.9°, 19.1°, and 20.9°±0.2 degrees 2θ, and also having any one or two peaks selected from 3.4° and 28.1°±0.2 degrees 2θ.

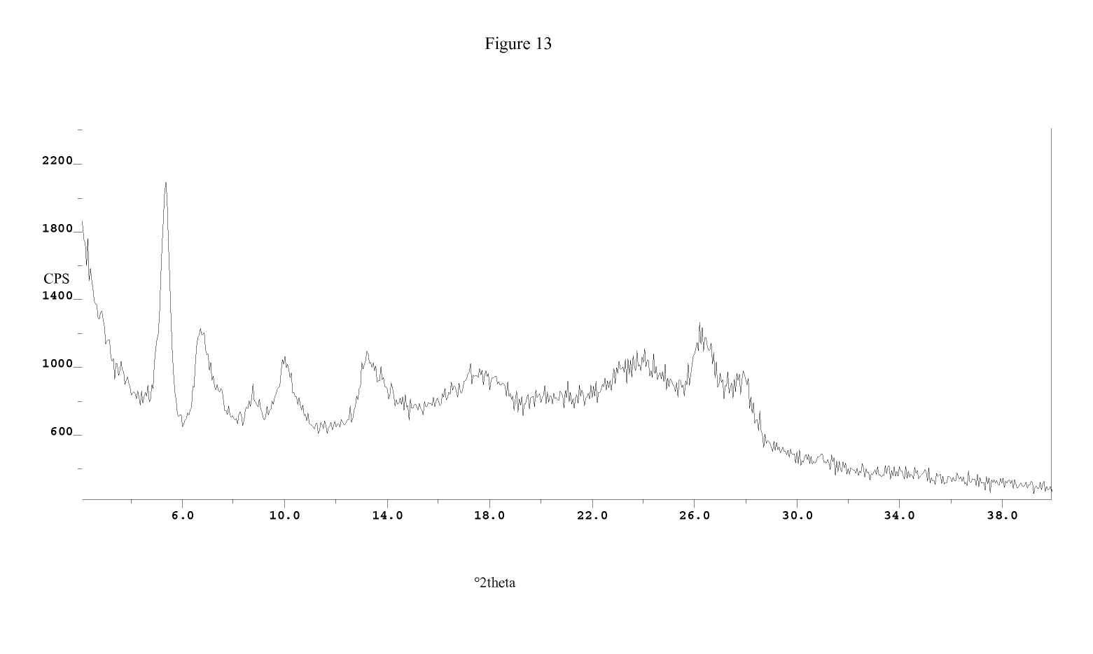

The present invention also provides a crystalline Linagliptin, designated Form XII. Form XII can be characterized by data selected from: a powder XRD pattern having peaks at 5.3°, 6.8°, 9.9°, 13.2°, and 26.3°±0.2 degrees 2θ; a powder XRD pattern as shown in FIG. 13; and any combinations thereof.

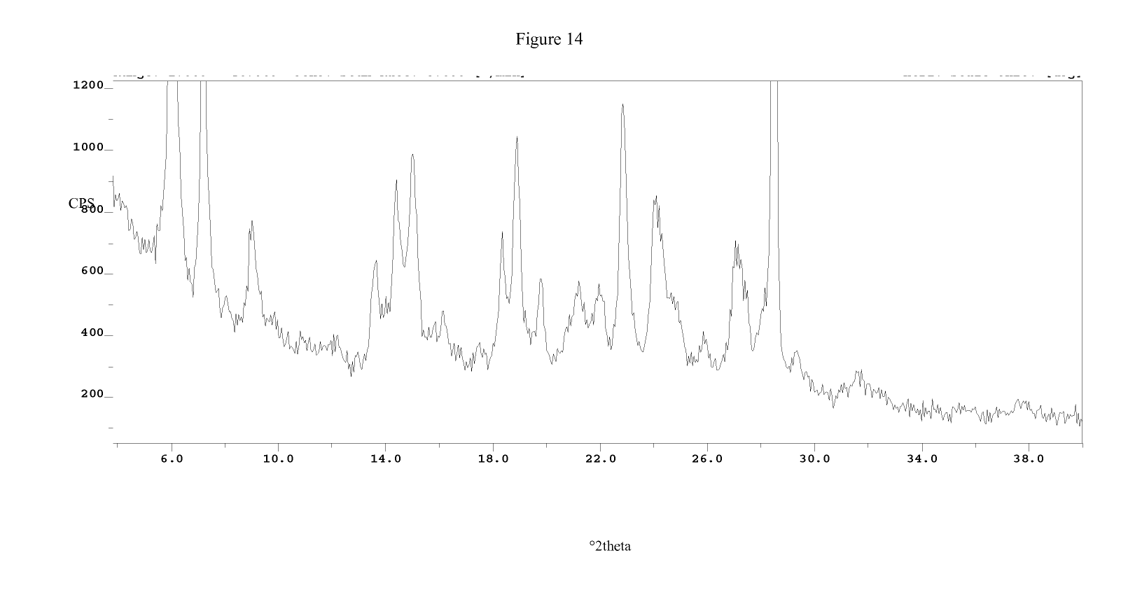

The present invention also provides a crystalline Linagliptin, designated Form XIII. Form XIII can be characterized by data selected from: a powder XRD pattern having peaks at 6.1°, 7.2°, 9.0°, 13.6°, and 14.4°±0.2 degrees 2θ; a powder XRD pattern as shown in FIG. 14; and any combinations thereof.

Alternatively, Form XIII can be characterized by a powder XRD pattern having peaks at 6.1°, 7.2°, 9.0°, 13.6°, and 14.4°±0.2 degrees 2θ, and also having any one, two, three, four, or five peaks selected from 15.1°, 19.0°, 19.8°, 22.9°, and 27.1°±0.2 degrees 2θ.

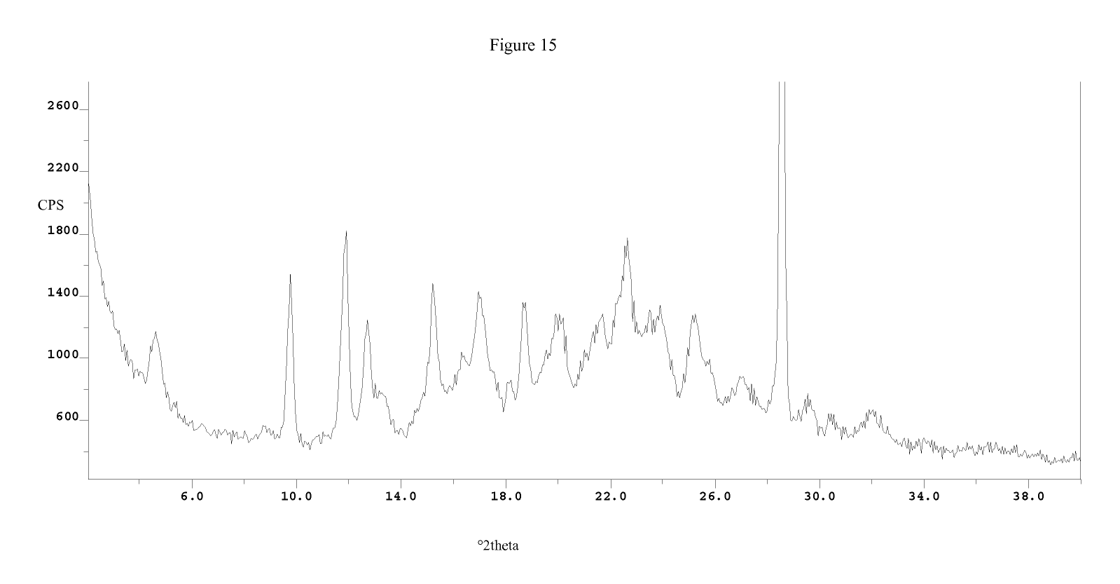

The present invention also provides a crystalline Linagliptin, designated Form XIV. Form XIV can be characterized by data selected from: a powder XRD pattern having peaks at 9.6°, 11.7°, 12.6°, 15.2°, and 16.9°±0.2 degrees 2θ; a powder XRD pattern as shown in FIG. 15; and any combinations thereof.

Alternatively, Form XIV can be characterized by a powder XRD pattern having peaks at 9.6°, 11.7°, 12.6°, 15.2°, and 16.9°±0.2 degrees 2θ, and also having any one, two, three, or four peaks selected from 18.6°, 19.9°, 22.5°, and 25.3°±0.2 degrees 2θ.

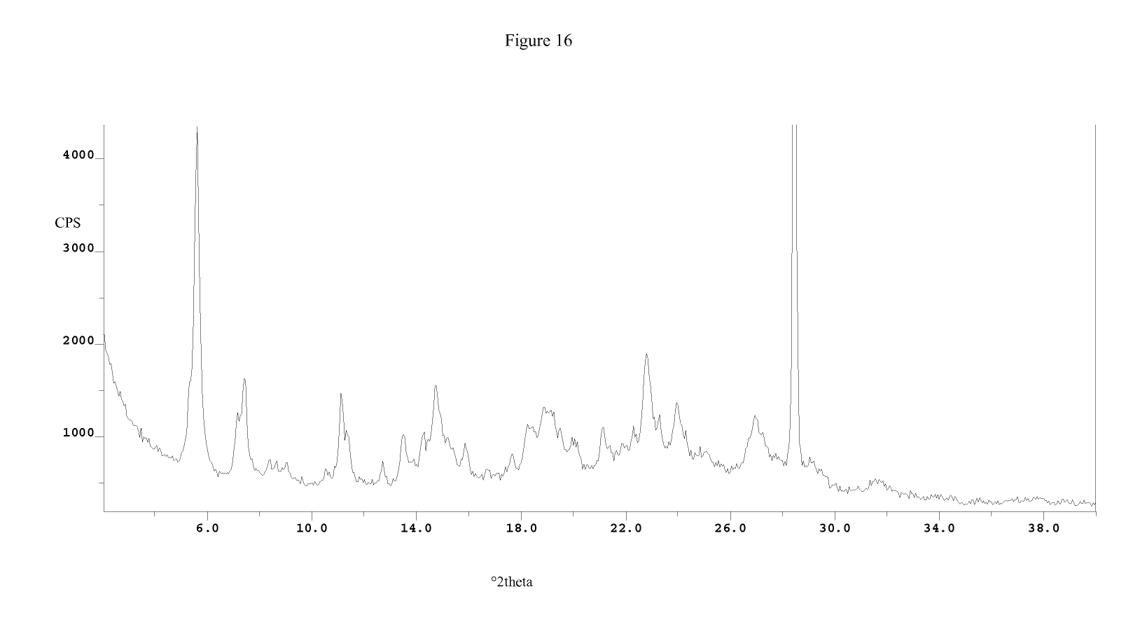

The present invention also provides a crystalline Linagliptin, designated Form XV. Form XV can be characterized by data selected from: a powder XRD pattern having peaks at 5.6°, 7.4°, 11.1°, 14.8°, and 22.8°±0.2 degrees 2θ; a powder XRD pattern as shown in FIG. 16; and any combinations thereof.

Alternatively, Form XV can be characterized by a powder XRD pattern having peaks at 5.6°, 7.4°, 11.1°, 14.8°, and 22.8°±0.2 degrees 2θ, and also having any one, two, three, four, or five peaks selected from 13.5°, 15.9°, 21.1°, 23.9°, and 26.9°±0.2 degrees 2θ.

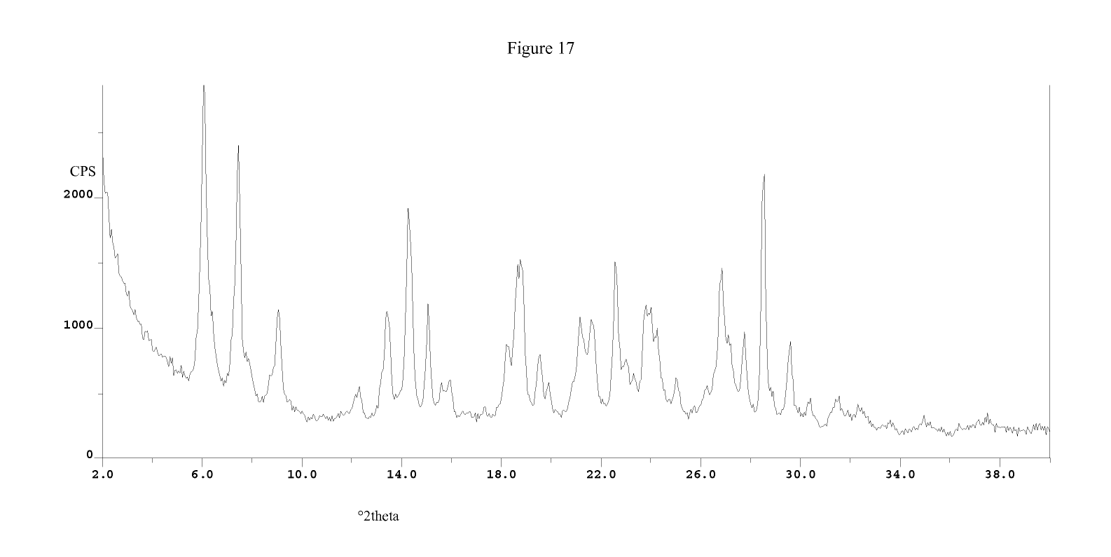

The present invention also provides a crystalline Linagliptin, designated Form XIII′. Form XIII′ can be characterized by data selected from: a powder XRD pattern having peaks at 6.1°, 7.4°, 13.4°, 14.3°, and 15.0°±0.2 degrees 2θ; a powder XRD pattern as shown in FIG. 17; and any combinations thereof.

Alternatively, Form XIII′ can be characterized by a powder XRD pattern having peaks at 6.1°, 7.4°, 13.4°, 14.3°, and 15.0°±0.2 degrees 2θ, and also having any one, two, three, four, or five peaks selected from 9.0°, 21.2°, 21.6°, 22.6°, and 26.8°±0.2 degrees 2θ.

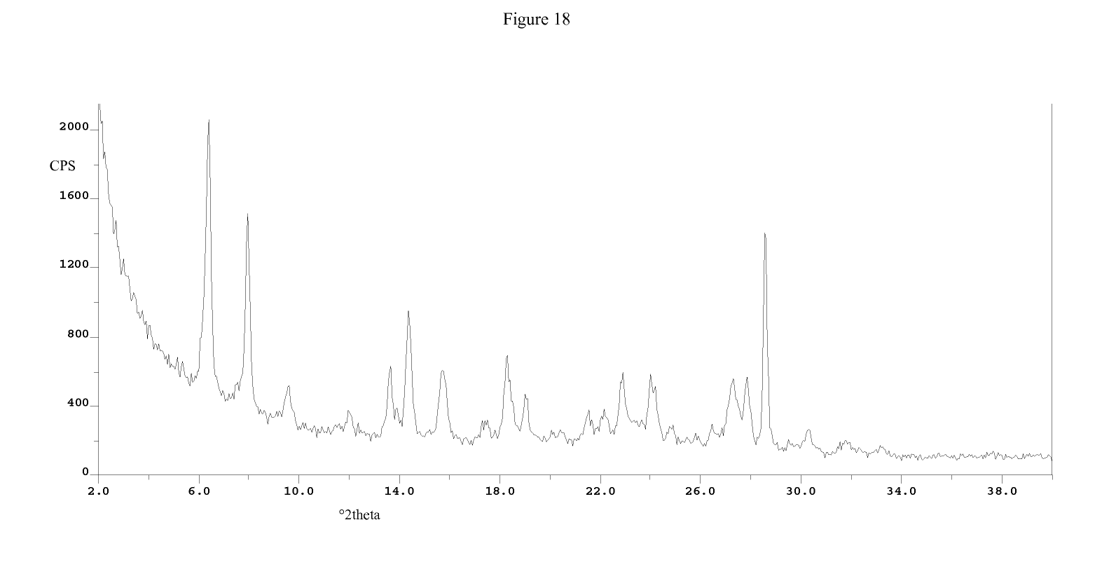

The present invention also provides a crystalline Linagliptin, designated Form XVI. Form XVI can be characterized by data selected from: a powder XRD pattern having peaks at 6.4°, 7.9°, 14.4°, 15.7°, and 18.3°±0.2 degrees 2θ; a powder XRD pattern as shown in FIG. 18; and any combinations thereof.

Alternatively, Form XVI can be characterized by a powder XRD pattern having peaks at 6.4°, 7.9°, 14.4°, 15.7°, and 18.3°±0.2 degrees 2θ, and also having any one, two, or three peaks selected from 19.0°, 22.9°, and 27.8°±0.2 degrees 2θ.

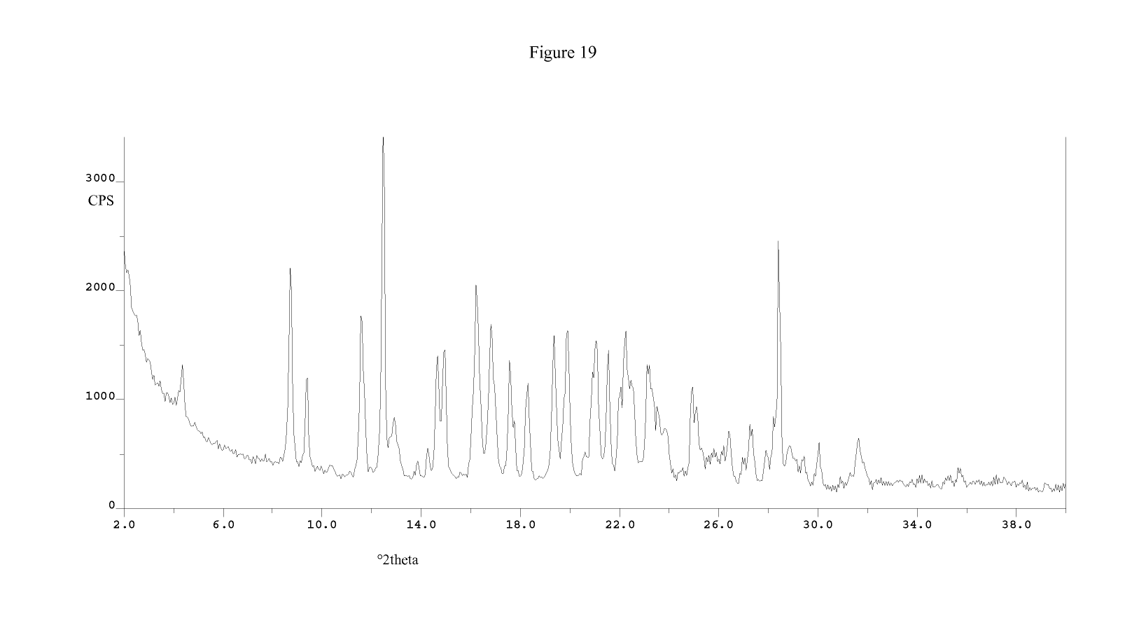

The present invention also provides a crystalline Linagliptin, designated Form VIII′. Form VIII′ can be characterized by data selected from: a powder XRD pattern having peaks at 8.7°, 9.4°, 14.9°, 16.2°, and 19.9°±0.2 degrees 2θ; a powder XRD pattern as shown in FIG. 19; and any combinations thereof.

Alternatively, Form VIII′ can be characterized by a powder XRD pattern having peaks at 8.7°, 9.4°, 14.9°, 16.2°, and 19.9°±0.2 degrees 2θ, and also having any one, two, three, four, or five peaks selected from 11.6°, 12.4°, 16.8°, 19.3°, and 21.5°±0.2 degrees 2θ.

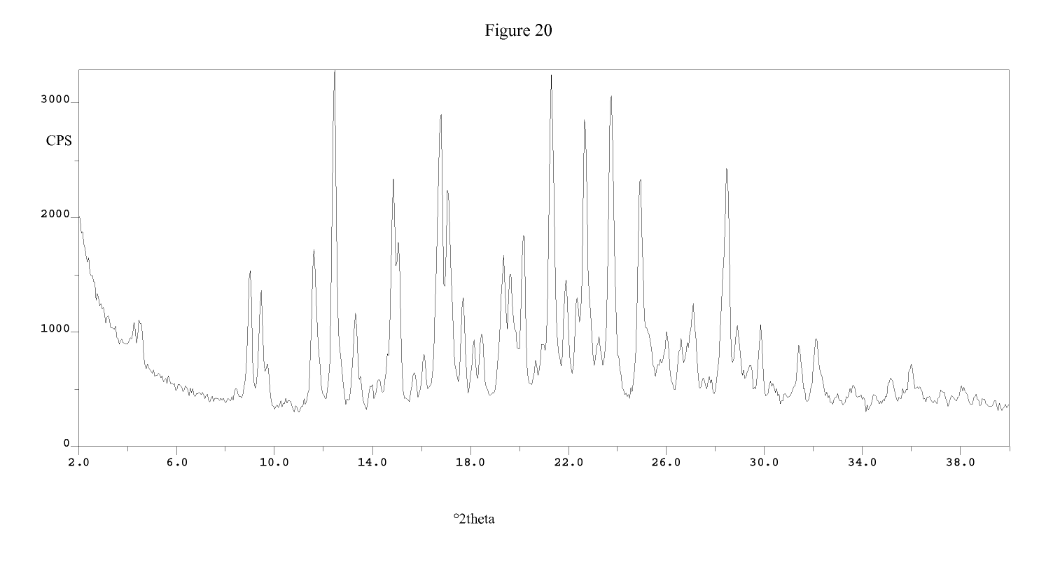

The present invention also provides a crystalline Linagliptin, designated Form XVIII. Form XVIII can be characterized by data selected from: a powder XRD pattern having peaks at 9.0°, 9.4°, 11.6°, 12.4°, and 16.7°±0.2 degrees 2θ; a powder XRD pattern as shown in FIG. 20; and any combinations thereof.

Alternatively, Form XVIII can be characterized by a powder XRD pattern having peaks at 9.0°, 9.4°, 11.6°, 12.4°, and 16.7°±0.2 degrees 2θ, and also having any one, two, three, four, or five peaks selected from 17.1°, 20.1°, 21.3°, 22.7°, and 23.7°±0.2 degrees 2θ.

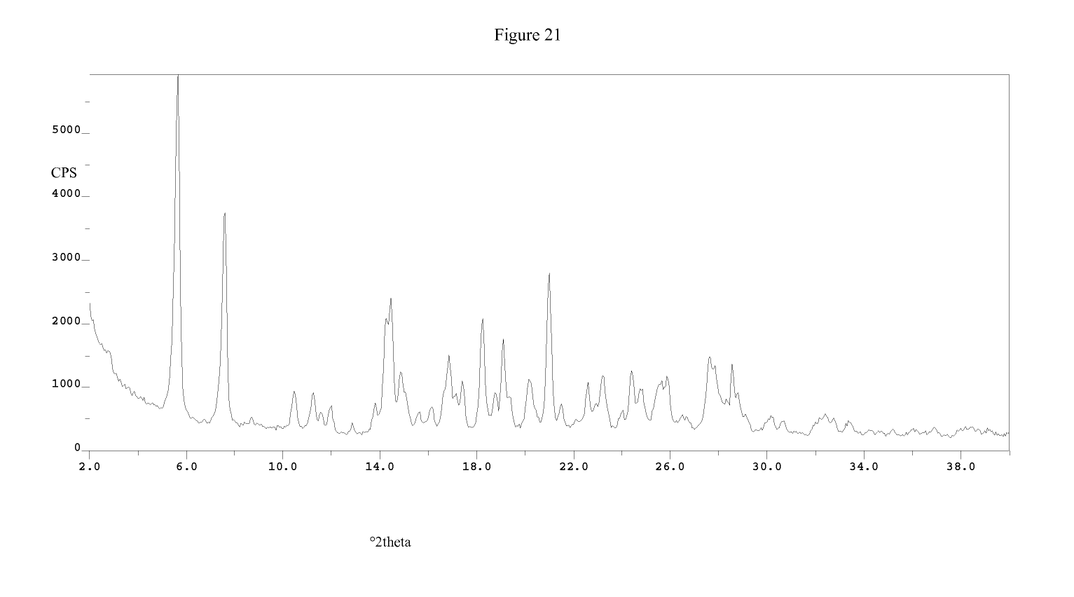

The present invention also provides a crystalline Linagliptin, designated Form XVII. Form XVII can be characterized by data selected from: a powder XRD pattern having peaks at 5.6°, 7.6°, 18.2°, 19.1°, and 21.0°±0.2 degrees 2θ; a powder XRD pattern as shown in FIG. 21; and any combinations thereof.

Alternatively, Form XVII can be characterized by a powder XRD pattern having peaks at 5.6°, 7.6°, 18.2°, 19.1°, and 21.0°±0.2 degrees 2θ, and also having any one, two, three, four, or five peaks selected from 10.4°, 14.4°, 16.8°, 23.2°, and 24.4°±0.2 degrees 2θ.

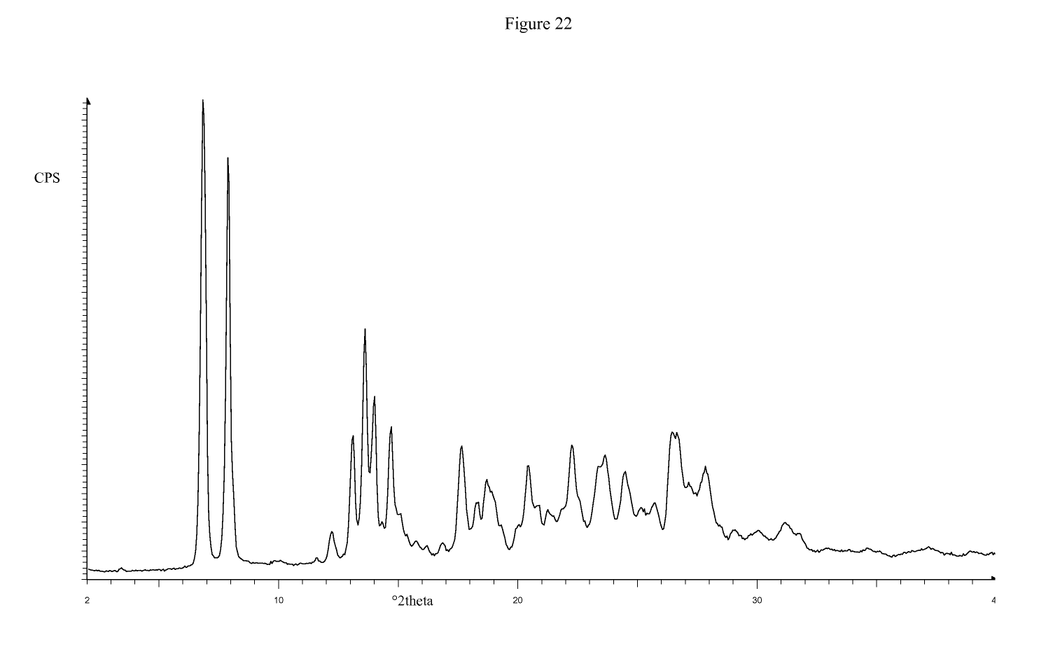

The present invention also provides a crystalline Linagliptin, designated Form XIX. Form XIX can be characterized by data selected from: a powder XRD pattern having peaks at 13.6°, 14.0°, 14.7°, 22.3°, and 26.5°±0.2 degrees 2θ; a powder XRD pattern as shown in FIG. 22; and any combinations thereof.

Alternatively, Form XIX can be characterized by a powder XRD pattern having peaks at 13.6°, 14.0°, 14.7°, 22.3°, and 26.5°±0.2 degrees 2θ, and also having any one or more additional peaks selected from 6.8°, 7.9°, 13.1°, 17.6°, and 24.5°±0.2 degrees 2θ.

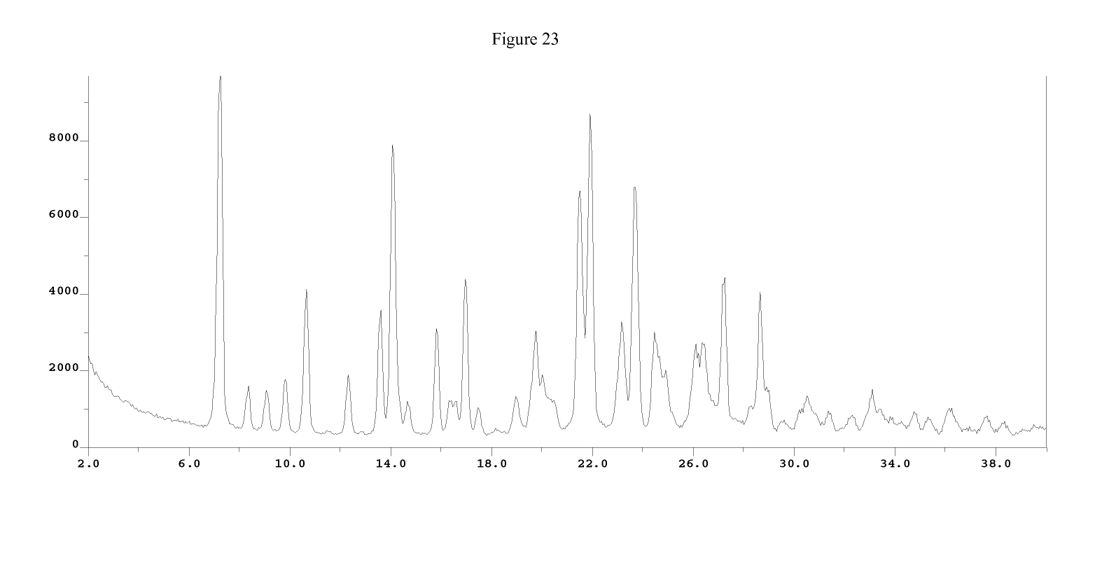

The present invention also provides a crystalline Linagliptin, designated Form XX. Form XX can be characterized by data selected from: a powder XRD pattern having peaks at 10.5°, 16.8°, 19.6°, 21.3°, and 21.7°±0.2 degrees 2θ; a powder XRD pattern as shown in FIG. 23; and any combinations thereof.

Alternatively, Form XX can be characterized by a powder XRD pattern having peaks at 10.5°, 16.8°, 19.6°, 21.3°, and 21.7°±0.2 degrees 2θ, and also having any one or more additional peaks selected from 7.0°, 8.9°, 9.6°, 12.1°, and 23.5°±0.2 degrees 2θ.

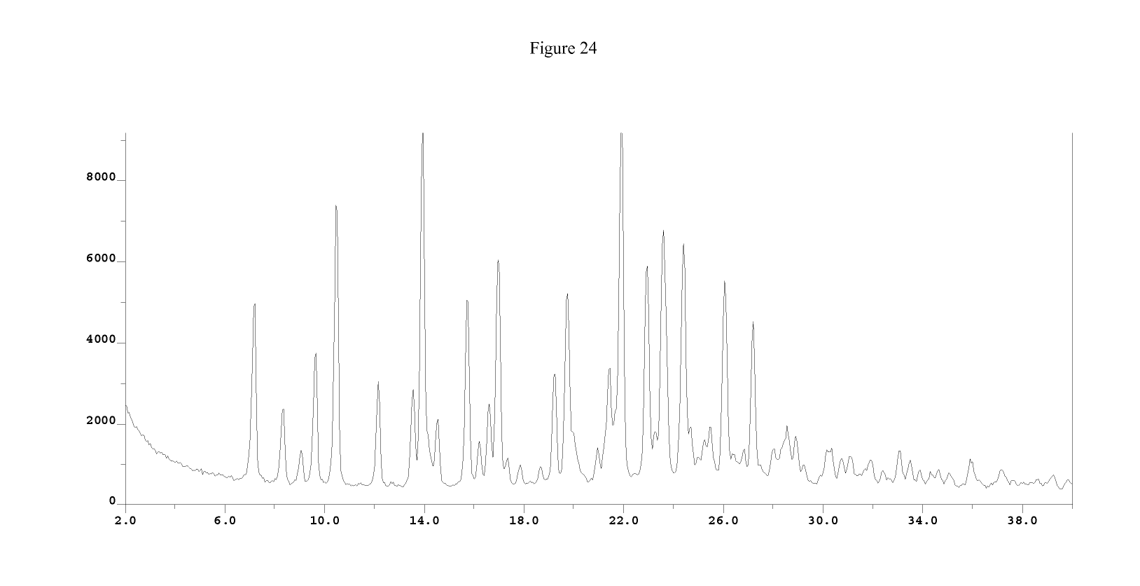

The present invention also provides a crystalline Linagliptin, designated Form XXI. Form XXI can be characterized by data selected from: a powder XRD pattern having peaks at 9.5°, 16.5°, 19.1°, 22.8°, and 26.0°±0.2 degrees 2θ; a powder XRD pattern as shown in FIG. 24; and any combinations thereof.

Alternatively, Form XXI can be characterized by a powder XRD pattern having peaks at 9.5°, 16.5°, 19.1°, 22.8°, and 26.0°±0.2 degrees 2θ, and also having any one or more additional peaks selected from 10.4°, 13.8°, 15.6°, 16.1°, and 21.8°±0.2 degrees 2θ.

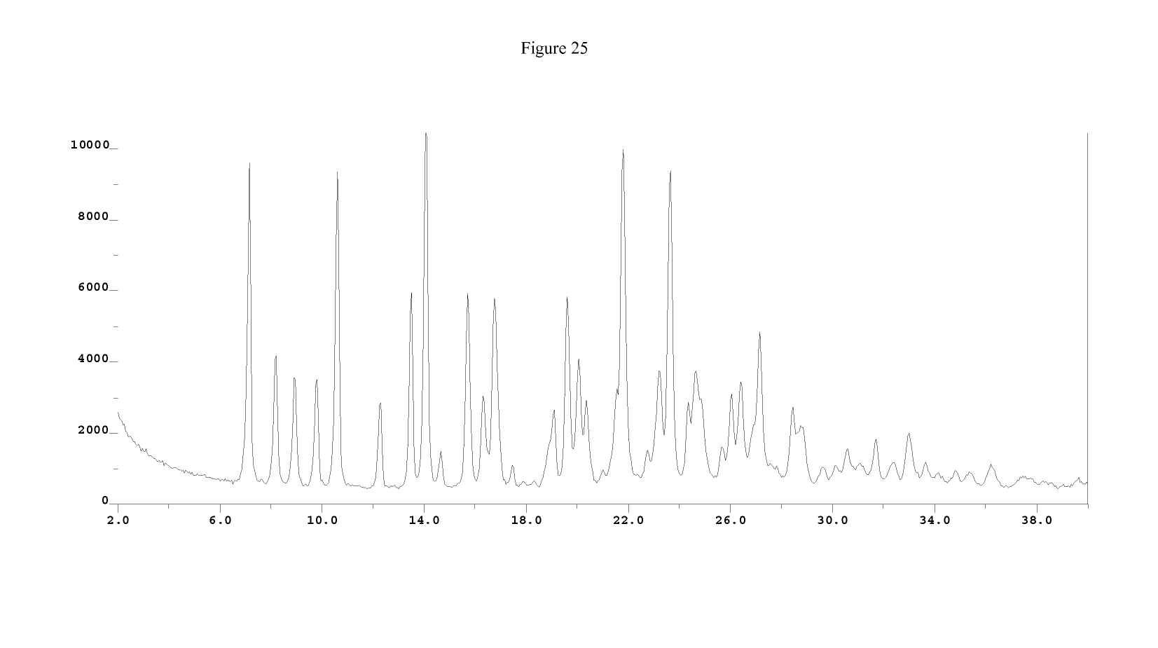

The present invention also provides a crystalline Linagliptin, designated Form XXII. Form XXII can be characterized by data selected from: a powder XRD pattern having peaks at 9.8°, 10.6°, 12.3°, 20.1°, and 23.7°±0.2 degrees 2θ; a powder XRD pattern as shown in FIG. 25; and any combinations thereof.

Alternatively, Form XXII can be characterized by a powder XRD pattern having peaks at 9.8°, 10.6°, 12.3°, 20.1°, and 23.7°±0.2 degrees 2θ, and also having any one or more additional peaks selected from 7.1°, 14.1°, 15.7°, 21.8°, and 27.2°±0.2 degrees 2θ.

The above mentioned Form XXII, may contain various solvents (organic and/or aqueous solvents) content. For example, the invention encompasses Form XXII having a total solvent content of about 5000 ppm to about 20 ppm; for example about 2500 ppm to about 20 ppm, for example about 1000 ppm to about 20 ppm, for example, about 500 ppm to about 20 ppm; for example, from about 200 ppm to about 50 ppm as measured by GC. The solvent may comprise, for example, acetonitrile, butanol, water or combinations thereof, or consist only of them.

In a specific embodiment, the crystalline Form XXII maybe a hydrate. For example, it may contain about 0.5% to about 10% w/w of water, as measured by KF.

In a specific embodiment, the invention encompasses Form XXII, as described above, can be non-hygroscopic.

Form XXII can be prepared, for example, by a process comprising crystallizing Linagliptin from a solution of acetonitrile, for example, by adding an anti-solvent such as n-heptane.

In addition to the properties mentioned above, Form XXII is particularly important as it can be obtained and exist at ambient conditions as single, pure phase. Form XXII is a polymorphic stable form, and do not transform to other forms, for example, when slurrying it in different solvents, such as acetonitrile and ethanol. Form XXII is also non-hygroscopic and polymorphic stable under different relative humidity levels. For example, when stored for 7 days at room temperature under 0-100% relative humidity, no polymorphic transformation is observed.

Form XXII also can also be used to prepare amorphous Linagliptin when heated to 100° C. Obtaining amorphous Linagliptin is important since amorphous material is more soluble in aqueous media. Preparing amorphous Linagliptin from Form XXII is also desirable since it is obtained at higher purity level.

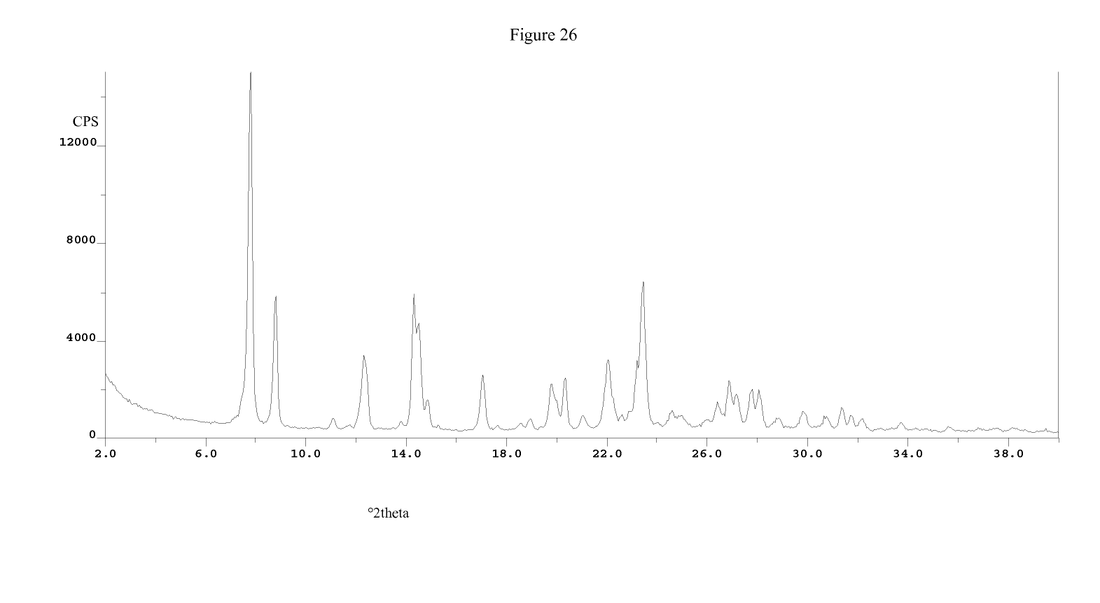

The present invention also provides a crystalline Linagliptin, designated Form XXIII. Form XXIII can be characterized by data selected from: a powder XRD pattern having peaks at 7.8°, 8.8°, 12.3°, 14.3° and 17.1°±0.2 degrees 2θ; a powder pattern as shown in FIG. 26; and any combinations thereof.

Alternatively, Form XXIII can be characterized by a powder XRD pattern having peaks at 7.8°, 8.8°, 12.3°, 14.3° and 17.1°±0.2 degrees 2θ, and also having any one or more additional peaks selected from 19.8°, 20.4°, 22.1°, 23.5°, and 26.9°±0.2 degrees 2θ.

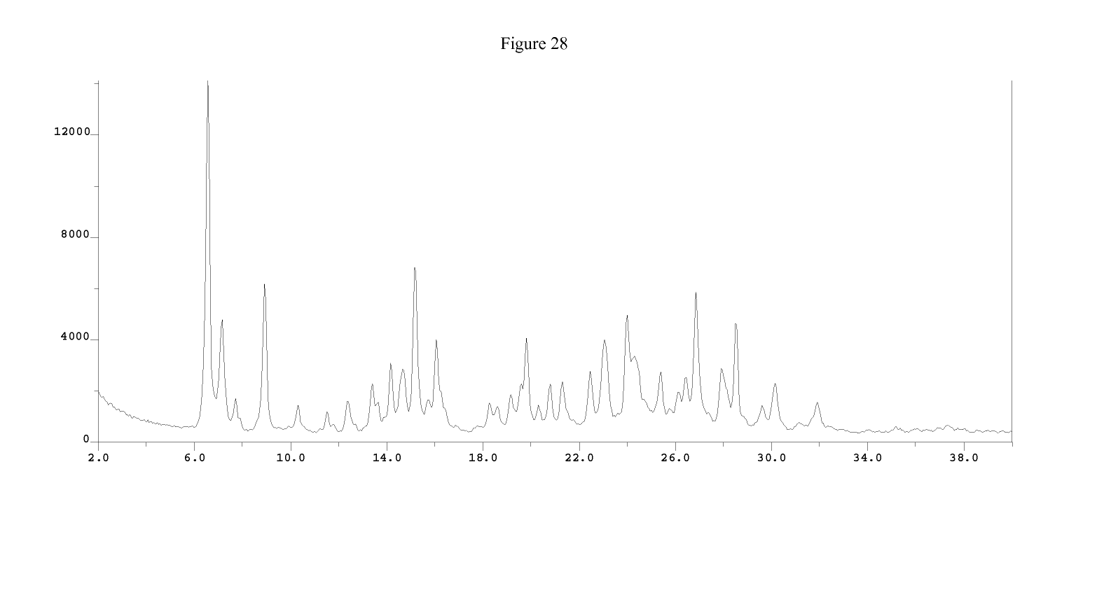

The present invention also provides a crystalline Linagliptin, designated Form XXIV. Form XXIV can be characterized by data selected from: a powder XRD pattern having peaks at 6.5°, 7.1°, 8.9°, 14.2°, and 15.2°±0.2 degrees 2θ; a powder pattern as shown in FIG. 28; and any combinations thereof.

Alternatively, Form XXIV can be characterized by the above listed peaks, and also by any one or more additional peaks selected from 16.0°, 19.8°, 20.8°, 21.3°, and 24.0°±0.2 degrees 2θ

The above described solid state forms of Linagliptin can be used to prepare different Linagliptin polymorphs, for example Forms A, B, C, D, and E, as described in WO 2007/128721; or they can be used to prepare Linagliptin salts, their solid state forms and formulations thereof.

For example, the present invention encompasses a process for preparing Linagliptin salts and solid state forms thereof comprising preparing any one or a combination of Linagliptin solid state forms of the present invention and converting them to a Linagliptin salt. The conversion can be done, for example, by a process comprising reacting any one or a combination of the above described Linagliptin solid state forms and an appropriate acid, to obtain the corresponding salt.

The present invention further encompasses a pharmaceutical composition comprising any one or combination of solid state Forms, as described above, and at least one pharmaceutically acceptable excipient and 2) the use of any one or combination of the above-described solid state Forms, in the manufacture of a pharmaceutical composition.

The present invention also encompasses a process for preparing a pharmaceutical composition comprising combining any one or more of the above mentioned forms of Linagliptin and at least one acceptable excipient.

The pharmaceutical composition can be useful for medicament, for example, for the treatment of type II diabetes.

The invention also provides a process to prepare formulation of Linagliptin comprising combining the above mentioned forms and at least one excipient.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### X-Ray Power Diffraction:

The XRD analyses were performed on ARL (SCINTAG) powder X-Ray diffractometer model X′TRA equipped with a solid state detector. Copper radiation of 1.5418 Å was used. Scanning parameters: range: 2-40 degrees two-theta; scan mode: continuous scan; step size: 0.05°, and a rate of 3 deg/min. The position of the silicon (111) peak was corrected to be 28.45 degrees two theta. The positions of the XRD peaks for the Linagliptin forms were corrected respectively.

GC:
Instrument: Agilent GC-7890A equipped with Agilent 7697A HeadSpace Sampler
Detector: FID
Column: Restek, Rxi-624Sil MS, 30 m*0.53 mm, 3 μm
Oven program: 40° C.-3 min; 15° C./min till 160° C.; 160° C.-1 min.
Flow: Helium, 5 ml/min
Split: 1:4
Inlet temp: 180° C.
Detector temp: 260° C.
Incubation conditions: 15 min at 80° C.
Analysis method: External standard.
Standards concentration:
Acetonitrile—equivalent to about 410 ppm
1-Butanol—equivalent to about 5000 ppm.
Sample preparation: About 100 mg sample in 1 ml Dimethylsulfoxide.

Karl-Fischer:
METTLER TOLEDO DL38 Karl Fischer Titrator,
with HYDRANAL—Composite 5 k as titrant

SSNMR Experimental Description:
Instruments parameters
13C NMR at 125 MHz using Bruker Avance II+500
SB probe using 4 mm rotors
Magic angle was set using KBr
Homogeneity of magnetic field checked using adamantane
Parameters for Cross polarization optimized using glycine
Spectral reference set according to glycine as external standard (176.03 ppm for low field carboxyl signal)

Scanning Parameters:
Magic Angle Spinning Rate:11 kHz
Delay time: 10 sec.
Number of Scans: 512 scans.

### EXAMPLES

### Example 1

### Preparation of Amorphous Linagliptin

Linagliptin (0.35 g) was partially dissolved in 10 ml of ethanol. The solvent was evaporated under reduced pressure to afford 0.30 g of amorphous Linagliptin.

### Example 2

### Preparation of Linagliptin Form I

Amorphous Linagliptin (25.1 mg) was weighed into a vial and 2,2,2-trifluoroethanol (0.125 mL) was added to the solid. The mixture was heated using a heat gun so that the solid dissolved. The solvent was allowed to evaporate slowly under a flow of nitrogen, and the resulting solid product was recovered to give Form I. The Form I product was then dried at 50° C. under vacuum for 24 hours to give Form A.

### Example 3

### Preparation of Linagliptin Forms II and III

Amorphous Linagliptin (25.8 mg) was weighed into a vial and 70/30 tetrahydrofuran/water (0.5 mL) was added to the solid. The mixture was heated using a heat gun so that the solid dissolved and then allowed to cool gradually to ambient temperature. The solvent was allowed to evaporate slowly under a flow of nitrogen and the resulting solid product was recovered to give Form II, which was then dried at 50° C. under vacuum for 24 hours to give Form III.

### Example 4

### Preparation of Linagliptin Form IV

Amorphous Linagliptin (50 mg) was weighed into a vial and methanol (0.1 mL) was added to the solid. The vial was sealed and the resulting slurry was shaken using a Heidolph Titramax 1000 platform which was linked to a Heidolph Inkubator 1000 as the temperature was cycled between ambient temperature and 40° C. every 4 hours. After 5 days the sample was removed and the slurry was re-heated using a heat gun so that the warm supernatant could be transferred to a new vial through a disposable syringe filter. The new vial was then sealed and the clear solution was placed in a freezer at −26° C. Within 24 hours a solid had precipitated and was allowed to dry by evaporation under nitrogen initially and then at 50° C. under vacuum to give Form IV.

### Example 5

### Preparation of Linagliptin Form V

A vial (20 ml) was charged with amorphous Linagliptin (300 mg) slurried in methanol:water 1:1 (1.8 ml) at room temperature. The mixture was stirred for 24 h at room temperature. The product was isolated by evaporation of the solvent. The wet product obtained was Linagliptin Form V. The wet product was dried in a vacuum oven at 40° C. overnight to obtain Linagliptin Form V.

### Example 6

### Preparation of Linagliptin Form VI

A vial (20 ml) was charged with amorphous Linagliptin (300 mg) slurried in acetone (0.9 ml) and water (0.9 ml) at room temperature. The mixture was stirred for 21 h at room temperature. The product was isolated by evaporation of the solvent. The wet product obtained was Linagliptin crystalline Form VI. The wet product was dried in a vacuum oven at 40° C. overnight to obtain Linagliptin Form V.

### Example 7

### Preparation of Linagliptin Form VII

A vial (20 ml) was charged with amorphous Linagliptin (300 mg) slurried in N,N-dimethyl formamide (0.9 ml) and water (0.9 ml) at room temperature. The mixture was stirred for 24 h at room temperature. The product absorbed all of the solvent, therefore water (about 2 mL) was added at room temperature, and the mixture was stirred for an additional20 h at room temperature. The product was isolated by vacuum filtration and washed with water (2×0.5 ml). The wet product obtained was Linagliptin crystalline Form VII.

### Example 8

### Preparation of Linagliptin Form B

A vial (3 ml) was charged with amorphous Linagliptin (200 mg) slurried in N,N-dimethyl acetamide (0.4 ml) at room temperature. The mixture was stirred for 24 h at room temperature. The product was then isolated by vacuum filtration and washed with N,N-dimethyl acetamide (0.25 ml). The wet product obtained was Linagliptin crystalline Form B.

### Example 9

### Preparation of Linagliptin Form V

A vial (20 ml) was charged with Linagliptin amorphous (300 mg) slurried in ethanol 95%:water 1:1 (1.8 ml) at room temperature. The mixture was stirred for 22 h at room temperature. The product was isolated by evaporation of the solvent. The wet product obtained was Linagliptin Form V.

### Example 10

### Preparation of Linagliptin Form V

A vial (20 ml) was charged with Linagliptin amorphous (300 mg) slurried in acetone (0.9 ml) and water (0.9 ml) at room temperature. The mixture was stirred for 21 h at room temperature. The product was isolated by evaporation of the solvent. The wet product obtained was Linagliptin crystalline Form VI. The wet product was then dried in a vacuum oven at 40° C. overnight to obtain Linagliptin Form V.

### Example 11

### Preparation of Linagliptin Form VIII

1-[(4-Methyl-quinazolin-2-yl)methyl]-3-methyl-7-(2-butyn-1-yl)-8-[(R)-3-(tert-butoxycarbonylamino)-piperidin-1-yl]-2,6-dioxo-2,3,6,7-tetrahydro-1H-purine (80 g, 0.14 mol) was mixed with dichloromethane (1.6 l, 20V), and trifluoroacetic acid (400 ml, 5V) to obtain a brown solution. The solution was stirred at room temperature for 3h. Then, water (400m1, 5V) was added, and the pH was adjusted to 8 with NH₄OH 25%. The organic phase was then separated, and concentrated to dryness to provide a yellow solid (92 g). The yellow solid was then mixed with ethanol (400 ml) at room temperature. The resulting brown solution turned into an off-white suspension after 5-10 min. The suspension was stirred for 1.5 h, and then filtered to provide a wet solid (143 g). The wet solid was dried for 15 h at 60° C. under vacuum to provide 8-[3(R)-Aminopiperidin-1-yl]-7-(2-butynyl)-3-methyl-1-(4-methylquinazolin-2-ylmethyl)xanthine Form VIII (69 g).

### Example 12

### Preparation of Linagliptin Form IX

1-[(4-Methyl-quinazolin-2-yl)methyl]-3-methyl-7-(2-butyn-1-yl)-8-[(R)-3-(tert-butoxycarbonylamino)-piperidin-1-yl]-2,6-dioxo-2,3,6,7-tetrahydro-1H-purine crystal form VIII (50 g, 0.106 mol) was mixed with 2-propanol (900 ml, 18V). The mixture was heated to reflux to form an off-white hot slurry. The slurry was stirred at reflux for 1 h and then cooled to RT over 1 h, and then further cooled to (0-5° C.) and stirred for 1 h at this temperature. The suspension was then filtered to provide a wet solid (76 g). The wet solid was dried for 18 h at 60° C. under vacuum to provide 8-[3(R)-aminopiperidin-1-yl]-7-(2-butynyl)-3-methyl-1-(4-methylquinazolin-2-ylmethyl)xanthine Form IX (48.82 g).

### Example 13

### Preparation of Linagliptin Form X

Amorphous Linagliptin (45 g, 0.095 mol) was mixed with 450 ml (10V) ethyl acetate. The mixture was heated to 40° C. to obtain an off-white hot suspension. The suspension was stirred over 15 h. The suspension was then cooled to room temperature, then filtered to provide a wet solid (64.5 g) which was dried 24 h at 60° C. under vacuum To provide Linagliptin Form X (43.6 g).

### Example 14

### Preparation of Linagliptin Forms XI and XI′

Amorphous Linagliptin (33 g, 0.07 mol) was mixed with methanol (132 ml, 4V), and the mixture was heated to 60° C. to obtain brownish solution. The solution was stirred over 15 min and then slowly cooled to RT. The resulting suspension was stirred at RT over 15 h, then filtered to provide a wet solid. The wet solid was dried 68 h at 60° C. under vacuum. The wet and dry solids were analyzed by XRPD and found to be new crystal forms XI′ and XI, respectively.

### Example 15

### Preparation of Linagliptin Form X

Linagliptin (45 g, 0.095 mol) was mixed with 450 ml (10V) ethyl acetate, and the mixture was heated to 40° C. to obtain an off-white hot suspension. The suspension was then stirred over 15 h. The suspension was then cooled to RT, then filtered to provide a wet solid (64.5 g) which was then dried at in a vacuum oven at 60° C. for 24 h to provide Linagliptin Form X (43.6 g).

### Example 16

### Preparation of Linagliptin Form X

Linagliptin (52 g, 0.11 mol) was mixed with 520 ml (10V) n-butyl acetate, and the mixture was heated to 40° C. to obtain off-white hot suspension. The suspension was stirred over 15 h and then cooled to room temperature and filtered To provide a wet solid (88 g), which was dried in a vacuum oven at 60° C. for 24 h to provide Linagliptin Form X (45 g).

### Example 17

### Preparation of Linagliptin Form XII

A sample of Linagliptin Form X was exposed to 100% relative humidity for 7 days. After 7 days the sample was analyzed by XRD and shown thereby to be Linagliptin form XII.

### Example 18

### Preparation of Linagliptin Form XIII

A vial (20 ml) was charged with amorphous Linagliptin (300 mg) slurried in 2-methyl tetrahydrofuran (3 ml) at RT. The slurry was heated to reflux. The solution formed was clear at 50° C. The solvent was evaporated overnight to form a residue. 2-Methyl tetrahydrofuran (1 ml) was added to the residue. The resulting mixture was stirred at reflux for 1 hour. The mixture was then cooled and the product was isolated by vacuum filtration. The wet product obtained was Linagliptin crystalline Form XIII. The product was dried in vacuum oven at 40° C. for 5 days to obtain Linagliptin crystalline Form XIII.

### Example 19

### Preparation of Linagliptin Form XIV

A vial (20 ml) was charged with Linagliptin amorphous (300 mg) dissolved in ethanol (1.5 ml) at RT. The mixture was heated to reflux. The solution formed was clear at room temperature. The mixture was stirred at reflux for 1 hour and then added diethyl carbonate (3 ml) in portions. The resulting solution was then cooled gradually to room temperature over 3.5 hours. Crystallization occurred at room temperature. The mixture was then stirred at room temperature for 20.5 hours and the crystalline product was collected by filtration. The product was dried in a vacuum oven at 40° C. for 24 hours to obtain Linagliptin crystalline Form XIV.

### Example 20

### Preparation of Linagliptin Form XV

A vial (20 ml) was charged with Linagliptin amorphous (1 g) slurried in 2-methyl tetrahydrofuran (10 ml) at RT. The slurry was heated to reflux to form a solution. The solution was stirred at reflux for 22.5 hours and then allowed to cool to room temperature. Crystallization occurred overnight. The product was isolated by vacuum filtration. The product was then dried in a vacuum oven at 40° C. for 21.5 hours to obtain Linagliptin crystalline Form XV.

### Example 21

### Preparation of Linagliptin Form XIII′

A vial (20 ml) was charged with Linagliptin amorphous (300 mg) mixed with methanol (1.0 ml) at RT. The mixture was heated to reflux to get a clear solution. Methyl-THF (2.0 ml) was added in portions. The solution was cooled gradually to room temperature over 2 hours. Methyl-THF (1.0 ml) was added to the resulting mixture. The mixture was then cooled to 2-8° C. Crystallization occurred after MTBE (9.0 ml) addition in portions. The mixture was maintained at 2-8° C. for about 22 hours to increase precipitation, and then filtered to obtain Linagliptin crystalline Form XIII′.

### Example 22

### Preparation of Linagliptin Form XVI

Prepared previously Linagliptin form XIII′ was dried in a vacuum oven at 40° C. over 24 hours to obtain Linagliptin crystalline Form XVI.

### Example 23

### Preparation of Linagliptin Form VIII′

Linagliptin (50.24 g, 0.106 mol) was mixed with EtOH (804 ml, 16V). The mixture was heated to reflux, then cooled to 50° C. MTBE (804 ml, 16V) was added to produce a brown solution, which was then cooled to (0-5)° C. An off-white precipitate formed. The mixture was stirred 1 h at same temperature, then filtered to obtain a wet solid (75 g), which was dried under vacuum (63 h at 60° C.) to provide Linagliptin Form VIII′ (44.16 g).

### Example 24

### Preparation of Linagliptin Form XVIII

A vial (20 ml) was charged with Linagliptin amorphous (300 mg) dissolved in acetonitrile (1.5 ml) at RT. The mixture was heated to reflux, and an additional 1.5 ml of acetonitrile was added to complete the dissolution. The mixture was stirred at reflux for 0.5 hour and then cooled gradually to room temperature over 5 hours. Precipitation occurred during the cooling. The mixture was then stirred at room temperature for about 22 hours. The product was isolated by vacuum filtration. The wet product obtained was Linagliptin crystalline Form XVIII. The product was dried in a vacuum oven at 40° C. for 24 hours to obtain dry Linagliptin crystalline Form XVIII.

### Example 25

### Preparation of Linagliptin Form XVII

A vial (20 ml) was charged with Linagliptin amorphous (300 mg) mixed with methyl-THF (3.0 ml) at RT. The mixture was heated to reflux, and additional methyl-THF (4.5 ml) was added. The mixture was stirred at reflux for about 24 hours and then cooled gradually to room temperature. The product was isolated by vacuum filtration. The wet product obtained was Linagliptin crystalline Form XVII. The product was dried in a vacuum oven at 40° C. for 24 hours to obtain dry Linagliptin crystalline Form XVII.

### Example 26

### Preparation of Linagliptin Form XIX

Linagliptin (95.1 g, 0.2 mol) was mixed with DCM (951 ml, 10V) at RT, and the mixture was evaporated to dryness. The obtained solid was dissolved in MeOH (951 ml, 10V) at RT, then evaporated to dryness to obtain Linagliptin crystal Form XIX (42.7 g).

### Example 27

### Preparation of Linagliptin Form XX

Linagliptin (52.2 g, 0.11 mol) was mixed with 260 ml (5V) EtOH at RT, and the mixture was heated to reflux. The mixture was then cooled fast from reflux to RT in an ice-water bath). The resulting suspension was stirred at RT for 1.5 hours, then filtered to provide wet Linagliptin crystal Form XX (42.7 g).

### Example 28

### Preparation of Linagliptin Form XXI

Linagliptin (1 g, 0.002 mol) was mixed in 5 ml (5V) of N-methyl formamide. The resulting mixture was stirred and heated to 105° C. till complete dissolution was observed. The solution was then cooled to room temperature and a solid precipitate formed. The solid was filtered and dried in a vacuum at 60° C. for 22 h. Linagliptin crystal Form XXI was obtained.

### Example 29

### Preparation of Linagliptin Form XXII

Linagliptin (1 g, 0.002 mol) was mixed in 20 ml (20V) of acetonitrile. The resulting mixture was stirred and heated to 90° C. until complete dissolution was observed. n-Heptane (20 ml, 20V) was added and the solution was cooled to room temperature. A solid precipitate formed and was filtered. Linagliptin crystal Form XXII was obtained.

### Example 30

### Preparation of Linagliptin Forms XX and XXII

Linagliptin (1 g, 0.002 mol) was mixed with 5 ml (5V) EtOH at RT. The resulting yellow solution turned into an off white suspension after 0.5 min. This suspension was stirred 1.5 h, then filtered. Wet Linagliptin crystal Form XX was obtained. The wet solid was dried 24 h at 60° C. under vacuum. Linagliptin crystal Form XXII was obtained.

### Example 31

### Preparation of Linagliptin Form XXIII

A sample of a polymorphic mixture of Linagliptin forms C and XI was heated in a TGA instrument according to the following conditions: 25-150° C., heating rate 10° C./min+15 minutes isotherm at 150° C. The product was confirmed by XRD analysis to be form XXIII.

### Example 32

### Preparation of Linagliptin Form XXII

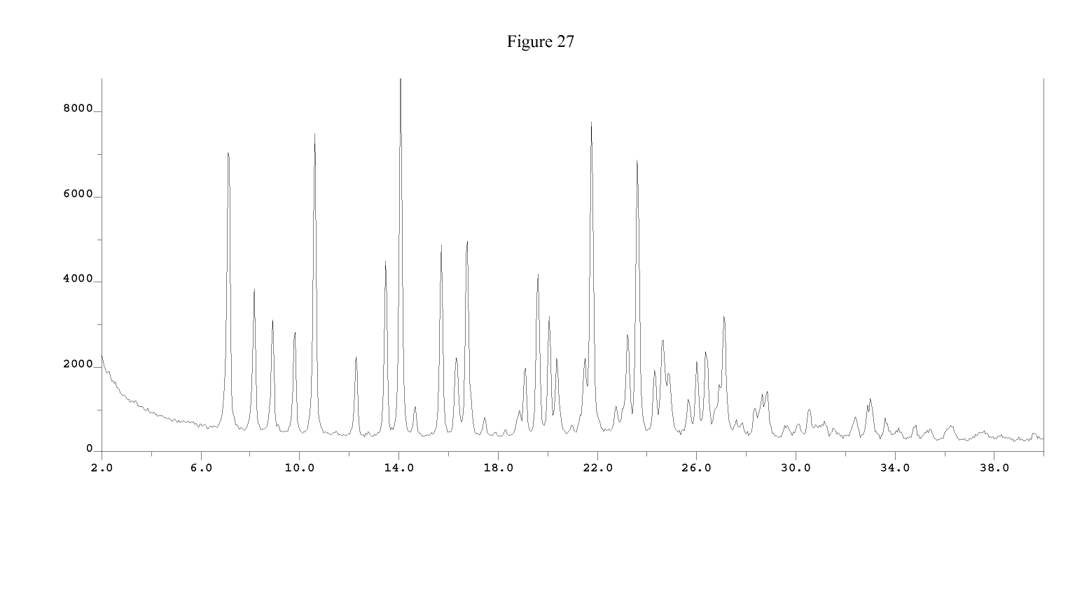

A sample of Linagliptin Form XXII was exposed to 100% RH at RT for 1 day. The sample was then dried in a vacuum oven at 30° C., overnight, to give Form XXII with less then 0.1% of residual solvents according to GC. The XRD of the product is shown in FIG. 27.

### Example 33

### Preparation of Linagliptin Form XXIV

Linagliptin (2.75 g, 5.8 mmol) was mixed with 27.5 ml (10V) toluene at 85° C., and then cooled to RT. The cooled mixture was seeded with Linagliptin Form XXII. The thus formed turbid suspension was stirred over 1 h at RT, than filtered and dried for 20 h at 40° C. in a vacuum oven to provide Linagliptin crystal form XXIV (1.45 g).

### Example 34

### Preparation of (R)-8-(3-amino-piperidin-1-yl)-7-(but-2-ynyl)-3-methyl-1-(4-methyl-quinazolin-2-ylmethyl)-3,7-dihydro-purine-2,6-dione (Form-XXII): A. 3-Methyl-7-(2-butyne-1-yl)-8-bromoxanthine

8-Bromo-3-methylxanthine was reacted with 1-bromo-2-butyne in the presence of base in a mixture of N-methyl pyrrolidone and toluene mixture. The reaction mixture was heated overnight. The reaction completion was determined, and the mixture was then cooled to ambient temperature. A solid precipitate formed on cooling precipitation. The product, 3-Methyl-7-(2-butyne-1-yl)-8-bromoxanthine, having greater than 95% purity was isolated by filtration and washed with toluene.

### Example 35

### Preparation of 8-bromo-7-(but-2-ynyl)-3-methyl-1-(4-methyl-quinazolin-2-ylmethyl)-3,7-dihydro-purine-2,6-dione

3-Methyl-7-(2-butine-1-yl)-8-bromoxanthine was reacted with 2-(chloromethyl)-4-methylquinazoline in the presence of base under phase transfer catalyst using a N-methyl pyrrolidone/toluene mixture as the reaction solvent. The reaction mixture was heated overnight. When the reaction was complete, the reaction mixture was cooled to ambient temperature. A solid precipitate formed and was separated by filtration and washed with toluene and then with water to provide the product, 8-bromo-7-(but-2-ynyl)-3-methyl-1-(4-methyl-quinazolin-2-ylmethyl)-3,7-dihydro-purine-2,6-dione having more than 97% purity.

### Example 36

### Preparation of (R)-8-(3-Amino-piperidin-1-yl)-7-(but-2-ynyl)-3-methyl-1-(4-methyl-quinazolin-2-ylmethyl)-3,7-dihydro-purine-2,6-dione (Form-XXII)

(R)-3-N-tert-Butoxycarbonylaminopiperidine was reacted with 8-bromo-7-(but-2-ynyl)-3-methyl-1-(4-methyl-quinazolin-2-ylmethyl)-3,7-dihydro-purine-2,6-dione in the presence of base. The reaction mixture was heated overnight. When the reaction was complete, the reaction mixture was cooled to ambient temperature. The cooled reaction mixture was washed several times with water and separated. The resulting 1-[(4-methyl-quinazolin-2-yl)methyl]-3-methyl-7-(2-butyn-1-yl)-8-[(R)-3-(tert-butoxycarbonylamino)-piperidin-1-yl]-2,6-dioxo-2,3,6,7-tetrahydro-1H-purine organic solution was greater than 95%. Purified by HPLC. An excess of aqueous HCl solution was added to the obtained 1-[(4-methylquinazolin-2-yl)methyl]-3-methyl-7-(2-butyn-1-yl)-8-[(R)-3-(tert-butoxycarbonylamino)-piperidin-1-yl]-2,6-dioxo-2,3,6,7-tetrahydro-1H-purine organic solution. The resulting mixture was stirred under heating until complete conversion was observed. Aqueous base was added to the reaction. The resulting mixture was stirred and separated. The organic phase was washed with aqueous base and separated. A non-polar or moderately polar solvent was added to the resulting organic phase. The mixture was partially concentrated to achieve precipitation, and the concentrated mixture was cooled and filtered to provide the wet crude product. The crude product was re-crystallized from alcohol, filtered and dried in vacuum oven with heating to afford dry solid Form-XXII of (R)-8-(3-amino-piperidin-1-yl)-7-(but-2-ynyl)-3-methyl-1-(4-methyl-quinazolin-2-ylmethyl)-3,7-dihydro-purine-2,6-dione having more than 98% purity.

What is claimed is:

1. Crystalline Form X of Linagliptin, characterized by data selected from: a powder XRD pattern having peaks at 8.3°, 9.6°, 13.0°, 17.6°, and 18.9°±0.2 degrees 2θ; a powder XRD pattern as shown in FIG. 10; and any combinations thereof.

2. The crystalline Form X according to claim 1, characterized by a powder XRD pattern having peaks at 8.3°, 9.6°, 13.0°, 17.6°, and 18.9°±0.2 degrees 2θ, further characterized by one, two, three, four, or five peaks selected from 8.8°, 10.6 13.6°, 16.2°, and 17.0°±0.2 degrees 2θ.

3. The crystalline Form X of any one of claims 1 and 2, which is anhydrous.

4. The crystalline Form X of any one of claims 1 to 3, which is non-hygroscopic.

5. Crystalline Form XXII of Linagliptin, characterized by data selected from: a powder XRD pattern having peaks at 9.8°, 10.6°, 12.3°, 20.1°, and 23.7°±0.2 degrees 2θ; a powder XRD pattern as shown in FIG. 25; and any combinations thereof.

6. The crystalline Form XXII according to claim 5, characterized by a powder XRD pattern having peaks at 9.8°, 10.6°, 12.3°, 20.1°, and 23.7°±0.2 degrees 2θ, and further characterized by one or more powder XRD peaks selected from 7.1°, 14.1°, 15.7°, 21.8°, and 27.2°±0.2 degrees 2θ.

7. The crystalline Form XXII of any one of claims 5 and 6, having a total solvent content of about 5000 ppm to about 20 ppm, as measured by GC.

8. The crystalline Form XXII of any one of claims 5 to 7, which is non-hygroscopic.

9. The use of the solid state forms of Linagliptin according to any one of claims 1 to 8 for the preparation of a different solid state form of Linagliptin.

10. The use of the solid state forms of Linagliptin according to any one of claims 1 to 8 for the preparation of a Linagliptin salt.

11. A process for preparing a Linagliptin salt, comprising reacting at least one of the crystalline forms according to any one of claims 1 to 8 with an acid.

12. A pharmaceutical composition comprising one or more crystalline forms according to any one of claims 1 to 8, and at least one pharmaceutically acceptable excipient.

13. The use of one or more crystalline forms according to any one of claims 1 to 8 for the manufacture of a medicament.

14. A process for preparing a pharmaceutical composition comprising combining one or more of the crystalline forms according to any one of claims 1 to 8 and at least one pharmaceutically acceptable excipient.
